(19) United States
(12) Patent Application Publication
(10) Pub. No.: US 2019/0244707 A1
(43) Pub. Date: Aug. 8, 2019

(54) EQUIPMENT MANAGEMENT SYSTEM

(71) Applicant: Stryker Corporation, Kalamazoo, MI (US)

(72) Inventors: David Terrance Becker, Grand Rapids, MI (US); John Thomas Shen, San Jose, CA (US); Rebecca Sue Paalman, Ada, MI (US); Matthew Todd Powell, Kalamazoo, MI (US); Daniel Joseph Martinson, Kalamazoo, MI (US); Frank Roland Caron, Paw Paw, MI (US)

(73) Assignee: Stryker Corporation, Kalamazoo, MI (US)

(21) Appl. No.: 16/316,382

(22) PCT Filed: Jul. 12, 2017
(86) PCT No.: PCT/US2017/041681

§ 371 Date: Jan. 9, 2019

Related U.S. Application Data
(60) Provisional application No. 62/361,221, filed on Jul. 12, 2016.

(51) Int. Cl. G16H 40/40; G16H 40/67; H04L 29/08

(52) U.S. Cl. CPC G16H 40/40; H04L 67/10; G16H 40/67

(57) ABSTRACT

A medical device management system includes a plurality of medical devices communicating with a local area network. A local network appliance forwards device data gathered from the medical devices to a cloud based service that processes and organizes the device data. The cloud based service may also be in communication with a sales database, an ERP system, a manufacturing database, and/or other databases. Users may request device data from the cloud based service using a conventional web browser. The device data may include location data, usage data, repair data, etc. In some cases, device data may come from a variety of sources and the cloud based service collates the multi-source data together into a set of records that form a digital replica of the actual medical device.

### CROSS-REFERENCE TO RELATED APPLICATIONS

This application claims priority to U.S. provisional patent application Ser. No. 62/361,221 filed Jul. 12, 2016, by inventors David Becker et al. and entitled EQUIPMENT MANAGEMENT SYSTEM, the complete disclosure of which is incorporated herein by reference.

### BACKGROUND

The present disclosure relates to an equipment management system, and more particularly to an equipment management system for a plurality of medical devices.

Medical facilities utilize large numbers of medical devices that come in a wide variety of different forms. These medical devices includes, but are not limited to, patient supports (e.g. beds, stretchers, operating tables, chairs, etc.); patient transport devices (e.g. wheelchairs, transport chairs, etc.); operating room equipment (e.g. drills, saws, sponges, lights, endoscopes, etc.); patient treatment devices (e.g. ventilators, respirators, DVT pumps, etc.); communication devices (e.g. cameras, nurse call communication equipment, pagers, etc.); pharmaceuticals (e.g. medicines, ointments, etc.); and still other types of devices. Tasks associated with the management of these devices, such as maintaining an accurate inventory, scheduling the use of the devices, locating the devices, repairing and/or servicing the devices, and other tasks) present substantial challenges to the administrators of the medical facility.

### SUMMARY

The present disclosure provides systems and methods for enabling more effective management of a medical facility's medical devices. In several embodiments, the present disclosure provides a cloud-based equipment management platform having a web-based user interface that enables authorized individuals at a medical facility to view in real-time, or near real-time, data regarding the medical devices within their facility. In various embodiments, the data viewable by the authorized individuals includes one or more off the following: an inventory of the medical devices in the possession of the medical facility; specific locations of those medical devices within the facility; the status of those medical devices (e.g. in service, out of service, clean, dirty, operational, non-operational, etc.); usage statistics of the medical devices; event histories for each of the medical devices; current software and hardware versions of the medical devices; and still other device data. The data may be viewed by multiple personnel at the medical facility using conventional a software application (e.g. a web browser) operating on any of a variety of different devices (desktop computers, laptops, tablets, smart phones, smart watches, Computers-on-Wheels (COWs), etc.) Further, the administrators of the medical facility can define profiles for different classes of authorized individuals so that only certain of the data is viewable by different classes of individuals (e.g. technicians, IT personnel, biomedical staff, nurses, doctors, janitorial staff, etc.).

According to one embodiment, a medical device management system for managing a plurality of medical devices is provided. The system includes a local network appliance, a user interface, and a cloud based service. The local network appliance is located at a medical facility and is adapted to receive device data from the plurality of medical devices. The cloud based service is located remotely from the medical facility and communicates with the local network appliance. The cloud based service receives the device data from the local network appliance. The user interface is displayed on a computer device located at the medical facility. The user interface is adapted to receive a data request from a user and to forward the data request to the cloud based service. The cloud based service identifies a set of the plurality of medical devices that are responsive to the data request and forwards to the user interface processed data regarding the set of medical devices. The processed data is based upon the device data.

According to another embodiment, a medical device management system is provided for managing a plurality of medical devices. The system includes a first local network appliance, a second local network appliance, a cloud based service, and a user interface. The first local network appliance is located at a first medical facility and adapted to receive device data from a first set of medical devices. The second local network appliance is located at a second medical facility and adapted to receive device data from a second set of medical devices. The cloud based service is located remotely from the first and second medical facilities and in communication with the first and second local network appliances. The cloud based service receives the device data from the first and second local network appliances. The user interface is displayed on a computer device. The user interface is adapted to receive a data request from a user and to forward the data request to the cloud based service. The cloud based service selects the first or second set of medical devices and forwards to the user interface processed data regarding the selected set of medical devices. The processed data is based upon the device data of the selected set of medical devices.

According to still another embodiment, a method is provided for managing a plurality of medical devices. The method includes receiving device data from a plurality of medical devices located at a medical facility; forwarding the device data to a cloud based service located remotely from the medical facility; receiving a data request from a user interface displayed on a computer device, wherein the data request relates to one or more of the medical devices at the medical facility; forwarding the data request to the cloud based service; identifying a set of medical devices from the plurality of medical devices that is responsive to the data request; processing the device data from the set of medical devices; and forwarding the processed device data to the user interface.

In still another embodiment, a medical device management system is provided that comprises a local network appliance, a cloud based service, and a sales database. The local network appliance is located at a medical facility and receives device data from a plurality of local medical devices. The cloud based service is located remotely from the medical facility and in communication with the local network appliance. The cloud based service receives the device data from the local network appliance. The sales database is in communication with the cloud based service and contains a first list of sales records identifying medical devices that were sold to the medical facility. The cloud base service compares the first list to a second list identifying the local medical devices that are in communication with the local network appliance and identifies any differences between the first and second lists.

According to still other embodiments, the cloud based service is adapted to provide a user interface to a computer device located at the medical facility. The user interface displays information regarding at least one of the differences. In some embodiments, the cloud based service identifies any of the local medical devices that are on the first list but not the second list and forwards the identified medical devices to the computer device.

In some embodiments, each of the medical devices includes a unique identifier and a transmitter. The transmitters transmit the device data and the unique identifier to the cloud based service through the local network appliance.

The local network appliance also sends location information to the cloud based service, in some embodiments. The location information may come from the medical devices themselves, the local network appliance, and/or an interface with a third party Real Time Locating System (RTLS). The location information identifies both the medical facility and a specific location within the medical facility.

In some embodiments, the user interface comprises a web-based software application and the computer device comprises at least one of the following devices: (1) a desktop computer, (2) a laptop computer, (3) a tablet computer, (4) a server, (5) a smart cell phone, (6) a smart TV, (7) virtual reality glasses, and (8) a smart watch. The user interface forwards the data request to the cloud based service by accessing a web page associated with the cloud based service. The user interface is also adapted to forward a user identity to the cloud based service which uses the user identity to filter the processed data. The user interface also allows, in at least one embodiment, a user to display a listing of the medical devices sorted by at least two different user-selectable criteria. The criteria include the following: a cumulative usage time of each of the medical devices, a time until a next servicing date for the medical devices, an age of the medical devices, a last known location of the medical devices, a last date of service of the medical devices, and still other criteria.

The device data sent by the medical devices includes one or more of the following parameters in at least one embodiment: (1) usage data indicating how long the medical devices have been in use; (2) event data indicating a time and a description of any events occurring with respect to the medical devices; and (3) current data indicating how much electrical current the medical devices have used.

The data request comprises a request for device data from a particular type of medical device, a request for a usage characteristic for a specific one of the medical devices, a request for an identification of medical devices within a particular location of the medical facility, or another type of request relating to the medical devices.

The cloud based service filters the processed data by correlating a user identification with a stored profile. The stored profile corresponds to a class of users and identifies what portion of the processed data is suitable for viewing by that class of users.

In some embodiments, the cloud based service updates a device record for each of the medical devices. The device record identifies a last known location of the medical device and a last date of service of the medical device. The device record is maintained and updated even if its corresponding medical device is subsequently moved to a different medical facility.

The cloud based server includes in some embodiments a device management server, a data repository server, and an application server. The device management server receives the device data from the plurality of medical devices and forwards the device data to the data repository server. The data repository server stores the device data and generates the processed data. The application server receives the processed data from the data repository server and forwards it to the computer devices on which the user interface is displayed. The local network appliance may communicate with both the device management server and the data repository server. In at least one embodiment, the local network appliance forwards the device data directly to the data repository server without forwarding the device data to the device management server.

The cloud based server may also or additionally be in communication with a manufacturing server. The manufacturing server includes manufacturing information regarding the manufacture of the plurality of medical devices. The processed data is based at least partially upon the manufacturing information.

In some embodiments, a first one of medical devices is adapted to transmit its device data to a second one of the medical devices. The second one of the medical devices is adapted to forward the device data of the first one of the medical devices to the local network appliance.

In still other embodiments, the cloud based service uses usage data and repair data gathered from multiple ones of the medical devices to predict when a medical device may need repair in the future. The cloud based service provides notification to the user interface when such a medical device is identified that may need future repair.

Before the various embodiments disclose herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a diagram of an equipment management system according to a first embodiment of the present disclosure;

FIG. 2 is a more detailed diagram of the equipment management system of FIG. 1;

FIG. 3 is a diagram of a representative medical device of the equipment management system of FIG. 1;

FIG. 4 is a diagram of the equipment management system of FIG. 1 showing in more detail an illustrative medical facility and examples of various types of medical devices that may be integrated into the equipment management system;

FIG. 5 is a diagram of the equipment management system of FIG. 1 showing in more detail an illustrative medical facility and various communication protocols that may be used in the medical facility;

FIG. 6 is a diagram of an illustrative digital device replica maintained by the equipment management system for each of the medical devices under management;

FIG. 7 is a screen shot displayable on one or more computer devices of the equipment management system showing an illustrative initial login page of a user interface;

FIG. 8 is a screen shot displayable on one or more computer devices of the equipment management system showing an illustrative master list of medical device categories within a medical facility;

FIG. 9 is a screen shot displayable on one or more computer devices of the equipment management system showing a detailed listing of each of the medical devices at the medical facility;

FIG. 10 is a screen shot displayable on one or more computer devices of the equipment management system showing an illustrative set of categories of batteries at the medical facility;

FIG. 11 is a screen shot displayable on one or more computer devices of the equipment management system showing an illustrative usage and service overview of the batteries at the medical facility;

FIG. 12 is a screen shot displayable on one or more computer devices of the equipment management system showing a detailed overview of an individual one of the batteries at the medical facility;

FIG. 13 is a screen shot displayable on one or more computer devices of the equipment management system showing a detailed listing of each of the batteries at the medical facility;

FIG. 14 is a screen shot displayable on one or more computer devices of the equipment management system showing an illustrative set of categories of hand pieces at the medical facility;

FIG. 15 is a screen shot displayable on one or more computer devices of the equipment management system showing an illustrative usage and service overview of the hand pieces at the medical facility;

FIG. 16 is a screen shot displayable on one or more computer devices of the equipment management system showing a detailed listing of each of the hand pieces at the medical facility;

FIG. 17 is a screen shot displayable on one or more computer devices of the equipment management system showing a detailed overview of an individual one of the hand pieces at the medical facility;

FIG. 18 is a screen shot displayable on one or more computer devices of the equipment management system showing an illustrative set of categories of beds at the medical facility;

FIG. 19 is a screen shot displayable on one or more computer devices of the equipment management system showing an illustrative usage and service overview of the beds at the medical facility;

FIG. 20 is a screen shot displayable on one or more computer devices of the equipment management system showing a detailed listing of each of the beds at the medical facility; and

FIG. 21 is a screen shot displayable on one or more computer devices of the equipment management system showing a detailed overview of an individual one of the beds at the medical facility.

### DETAILED DESCRIPTION OF THE EMBODIMENTS

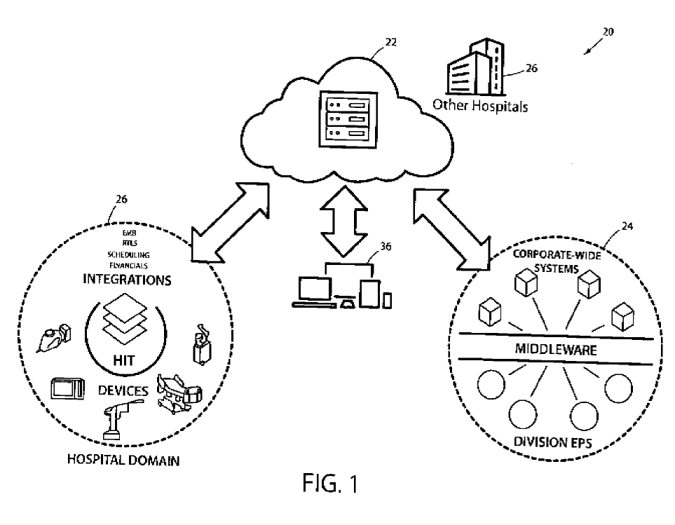

An equipment management system 20 according to one embodiment of the present disclosure is shown in diagram form in FIG. 1. Equipment management system 20 is specifically designed for use in managing devices used at one or more medical facilities, such as hospitals, clinics, and the like. Although equipment management system 20 will be described herein with respect to medical devices, it will be understood that system 20 can be applied to non-medical devices as well. Further, although the following description focus on only a few types of medical devices, such as surgical hand pieces, batteries, and beds, it will be understood that system 20 is designed to be used with a wide variety of additional and/or different types of medical devices. Such additional medical devices includes, but are not limited to, other types of patient supports (e.g. stretchers, operating tables, chairs, etc.); patient transport devices (e.g. wheelchairs, transport chairs, etc.); operating room equipment (e.g. drills, saws, sponges, lights, endoscopes, etc.); patient treatment devices (e.g. ventilators, respirators, DVT pumps, etc.); communication devices (e.g. cameras, nurse call communication equipment, pagers, etc.); pharmaceuticals (e.g. medicines, ointments, etc.); and still other types of medical devices.

Equipment management system 20 is designed to provide administrators of a medical facility with an up-to-date overview of the status of all of the medical devices being managed by system 20. The status information provided by system 20 includes information about the usage of the medical devices, the maintenance history of the medical devices, outputs from one or more sensors of the medical devices, sales information about the medical devices, the current and past operational status of the medical devices, and, in some embodiments, the last known location of each of the medical devices. Equipment management system 20 is further designed to allow the administrators to sort and filter the data from the monitored medical devices so that administrators can quickly focus on only the particular data that is of interest to them at a given moment. For example, if an administrator wishes to know how many of a particular type of endoscopes are located at a given facility, and/or the usage or current status of those endoscopes, equipment management system 20 is designed to allow the user to easily request only this data. In response, system 20 automatically filters out all of the other medical device data that has been gathered by system 20.

Equipment management system 20 is also designed, in some embodiments, to provide notifications or suggestions to the administrator of the medical facility that improve the medical facility's internal procedures and/or management of their equipment. For example, equipment management system 20 is designed to inform the administrators of the medical facility when particular medical devices are showing unusual amounts of use or disuse. This allows the administrators to investigate the reasons for such overuse or underuse and take steps to more evenly distribute the equipment usage. As another example, management system 20 provides advance notifications to the administrators when equipment should be serviced. System 20 also provides a unified location for receiving error alerts from the medical devices being actively managed by system 20. Still further, in at least some embodiments, system 20 is configurable to compare the list of the actively managed medical devices with the sales records from the vendor or vendors of the medical devices, and to identify discrepancies between the equipment the medical facility purchased and the equipment that is on-site at the medical facility. In still other embodiments, system 20 is adapted to predict when servicing of equipment will be likely outside of the regularly scheduled servicing using statistical analyses of the usage, repair, and/or error data gathered from a statistically significant number of similar-type medical devices. In still other embodiments, system 20 is adapted to perform still other features, as will be described in greater detail below.

In addition to providing all of the foregoing data to the medical facility administrators, equipment management system 20 is designed to allow the administrators to grant selective access of this information to other personnel at the medical facility, such as doctors, nurses, technicians, biomedical engineers, sales personnel, etc. Some or all of the data collected by equipment management system 20 is also available to be viewed by authorized agents of the vendor of equipment management system 20.

As shown in FIG. 1, system 20 includes a management service 22, one or more vendor's enterprise systems 24, and one or more medical facilities 26. The management service 22 is a cloud-based service that is accessible via the Internet. The vendor's enterprise system 24 is the enterprise system of the vendor of the medical devices that are being managed by system 20. The medical facility 26 is a hospital, clinic, or other type of health care institution in which the medical devices being managed by system 20 are located.

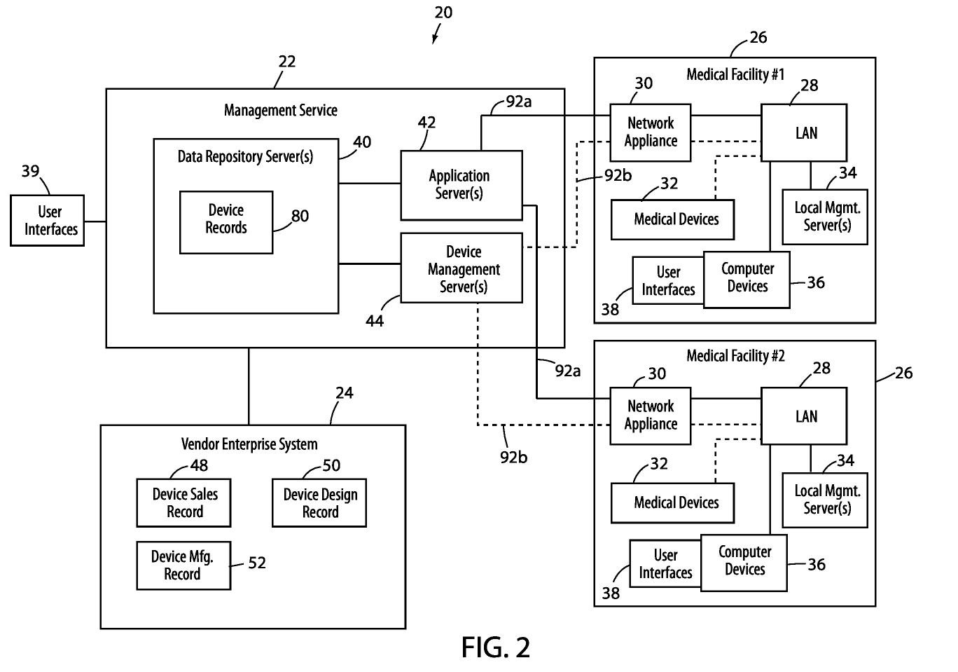

A more detailed view of the structures of equipment management system 20 is shown in FIG. 2. As can be seen therein, management service 22 communicates with a local area network (LAN) 28 associated with each medical facility 26. More specifically, each LAN 28 includes one or more network appliances 30 that allow the devices on the LAN 28 to communicate externally of the medical facility. In some embodiments, network appliances 30 are conventional edge routers or subscriber routers that couple the medical facility's LANs 28 to the Internet via one or more Internet Service Providers or Network Service Providers. Gateways 30 may include a built-in firewall, or the firewall may be implemented separately from the network appliance 30. In some LANs 28, there may be no firewall.

Each LAN 28 includes a plurality of medical devices 32 that are in communication with LAN 28. Medical devices 32 communicate with LAN 28 in a variety of different manners, as will be discussed in greater detail below. Medical devices 32 forward device data about themselves through either network appliance 30 for forwarding to management service 22, or to a local management server 34 that is likewise coupled to LAN 28. When the device data is forwarded to the local management server 34, the local management server 34 collects the device data, processes the device data in one or more manners that will be discussed in greater detail below, and forwards some or all of the processed data to management service 22 via network appliance 30.

Management service 22 collects all of the device data that is forwarded to it from the network appliances 30 of one or more medical facilities 26. Although FIG. 2 illustrates system 20 having two medical facilities 26, it will be understood by those skilled in the art that this is merely for purposes of illustration. System 20 can be implemented with only a single medical facility 26 in at least one embodiment. In other embodiments, system 20 can be implemented with more than two medical facilities 26 that communicate with management service 22.

In addition to collecting all of the device data sent from the various medical facilities 26, management service organizes the received device data according to which medical facility the data was received, as well as from which individual medical device 32 the device data was generated. Management service 22 also maintains and updates device records for each medical device 32. When new data is received from a particular medical device 32 via a network appliance 30, management service 22 updates the corresponding portion or portions of the device record for that particular medical device 32.

Management service 22 also analyzes and processes the device data received from the medical devices 32 and forwards the selected components of that information to one or more computer devices 36 that are in communication with management service 22. The computer devices 36 are shown in FIG. 2 as being positioned within the medical facilities 26, but this is merely one example of the potential locations for computer devices 36. Computer devices 36 may be positioned outside of medical facilities 26 at any location where they have network access to management service 22, which, as noted, is a cloud based service that is accessible via the Internet.

Each computer device 36 includes a local user interface 38, that may comprise a display and/or touch screen in combination with one or more tools for manipulating and/or responding to the information displayed on the display or touchscreen. Such tools include, but are not limited to, a mouse, a keypad, a trackball, a stylus, and the like. Computer devices 36 may be conventional desktop computers, laptop computers, tablet computers, Computers-On-Wheels (COWs), smart cell phones, or other types of computers. It is not necessary for computer devices 36 to be of a uniform type within any given management system 20. Thus, for example, some computer devices 36 may be smart phone while some other ones may be desktop computers. Other variations are, of course, possible.

In the embodiment shown in FIGS. 1 and 2, management service 22 is a web-based service that is accessible to users of computer devices 36 via one or more conventional world wide web browser applications, such as, but not limited to, Microsoft's Internet Explorer browser, Google's Chrome browser, Mozilla's Firefox browser, Apple Computer's Safari browser. Other types of applications executed by computer devices 36 may alternatively be used for accessing the services of management service 22 via computer devices 36.

In the embodiment shown in FIG. 2, management service 22 is made up of a collection of three different types of servers: one or more data repository servers 40, one or more application servers 42, and one or more device management servers 44. Servers 40, 42, and 44 are all in communication with each other, though not necessarily physically located at a common location. In some embodiments, servers 40, 42, and 44 are coupled to each other as part of a local area network that is coupled via a network appliance, or other device, to the Internet. In other embodiments, one or more of servers 40, 42, and 44 are coupled to each other via the Internet or other wide area network connection. When located in different countries or political regions from each other or from one or more of medical facilities 26, system 20 is designed to take into account the legal rules, if any, that may restrict the movement of data collected from medical devices 32 across the boundaries between the different regions. In some embodiments, system 20 strips out of its messages any data that is prohibited from crossing such boundaries. When possible, system 20 stores collected information in geographic locations that have no such restrictions, or a minimal amount of such restrictions.

Application server 42 communicates with the computer devices 36 and acts as an interface between the computer devices 36 and data repository server 42. Application server 42 may host one or more other applications besides those associated with equipment management system 20. Such applications utilize the existing infrastructure of system 20, but perform one or more different functions from equipment management system 20.

Device management server 44 communicates with the medical devices 32 and acts as an interface between the medical devices 32 and data repository server 42. The communication paths between a facility's network appliance 30 and maintenance service 22 are different. Specifically, as shown in FIG. 2, the communication between computers 36 and management service 22 follows a first communication path 92a, while the communication between the medical devices 32 and the facility's network appliance 30 follows a second path 92b.

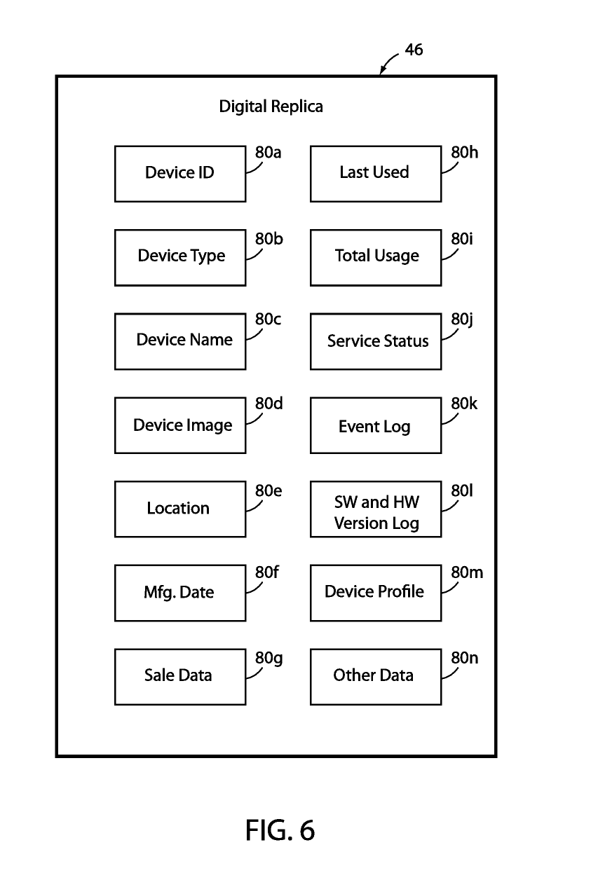

Data repository server 40 maintains one or more digital records 80 which, in combination, make up digital replicas 46 of each medical device 32 (FIG. 6). More specifically, data repository server 40 maintains at least a device profile record 80m for each medical device 32 that reports device data via network appliances 30, as well as each medical device 32 that is identified in vendor enterprise system 24. The device profile record 80m contains information of where the rest of the records 80 that comprise the digital replica 46 for each device are located. In some instances, some or all of these records 80 are stored in data repository 40. However, in some instances, the various records 80 of digital replicas 46 are stored in disparate locations and the device profile records 80m keeps track of, and update, the locations of each of these records 80. Each device profile 80m and digital replica 46 includes a unique ID that corresponds to an associated physical device 32. The various records 80 of each digital replica 46 also server as the repositories for the incoming device data that is forwarded to management service 22 from the medical facilities 26 and from the vendor's enterprise system 24.

The device data that is sent from the medical facilities 26 to the records 80 of the digital replicas 46 includes, but is not limited to, one or more of the following: the times and dates at which the medical devices 32 have been used; the cumulative amount of usage of the medical devices 32; the times, dates, and types of servicing of the medical devices 32; any error codes generated by the medical devices 32, as well as the times and dates of the error codes; one or more battery statistics of those medical devices 32 that are battery powered (e.g. current battery charge level, current battery drain rate, date and time of last discharge, current voltage, resistance, etc.); outputs from any one or more sensors that are included on particular ones of the medical devices 32, such as temperature sensors, current sensors, light sensors, speed sensors, force sensors, pressure sensors, etc.; the times, dates, and amounts of motor usage for those medical devices 32 having one or more motors; and the current software and hardware version installed on the medical device 32. In some embodiments, the local management server 34 is able to detect specific locations of the medical devices 32 within a given medical facility 26, either in conjunction with locating equipment that is sold by the same vendor as the vendor of equipment management system 20, or in conjunction with 3^{rd} party locating systems that communicate with local management server 34. Still other device data may also be sent from the medical facilities 26 to the management service 22 and entered in the individual digital replicas 46.

The device data that is sent from the vendor's enterprise system 24 includes, but is not limited to, data selected from one or more of the following sets of records: a set of device sales records 48, a set of device design records 50, and/or a set of device manufacturing records 52. The device sales records 48 include one or more of the following: the unique IDs of the medical devices 32; the make and model numbers of the medical devices 32; a type and/or description of each medical device 32; the original purchasers of the medical devices 32; subsequent purchasers of the medical devices 32; and the location of the original purchasers and subsequent purchasers. The purchaser identification and location information includes information identifying which medical facility 26 the medical device 32 is associated with. When available, this information specifies not only the address of the purchaser, but also which campus, building, and/or other location information indicating where the medical devices 32 are expected to be located.

The device design records include any one or more of the following: engineering data regarding the design of the medical device, such as technical drawings, operational parameters, etc.; operational manuals; maintenance manuals; technical notes; testing results; and any other design-related information. The device design records include not only records corresponding to the design of the type of device, but also individual design history files for the individual instances of a particular medical device 32. In other words, if a particular medical device 32 is a bed, for example, the device design records will contain records that are common to all of the beds that are of the same model or type, as well as any records that correspond to that particular bed. Such individual records may include, as but one example, configuration data for one of more sensors that are installed on a particular bed. Still other individual records may also be included within the device design records.

The device manufacturing records 52 include one or more of the following sets of records: when and where the medical devices 32 were manufactured; an identification of which options, if any, were installed or built into each medical device 32; the unique identifiers (if any) of the components or subcomponents of each medical device 32 (e.g. a particular type of siderail of a bed 32 may have a unique ID, or a particular electronic circuit board may have a unique ID); the current software and/or hardware version that is installed on the medical devices 32; and a log of the software and/or hardware updates to the medical devices 32. In addition to the foregoing data, the vendor's enterprise system 24 may also include any one or more of the following: a recommended service schedule for each of the medical devices 32, including an identification of the recommended times of service and the recommended types of service; information regarding FDA compliance and/or certification; material safety data sheets, if applicable; and software, hardware, and/or firmware updates for the medical devices 32. Still other device data may also be sent from the vendor's enterprise system 24 to the management service 22 for entry into the individual digital replicas 46 of devices 32.

FIG. 6 illustrates in greater detail one example of the types of data records and data that are maintained in an individual digital replica 46 for a particular medical device 32. As shown therein, digital replica 46 includes a device ID record 80a, a device type record 80b, a device name record 80c, a device image record 80d, a location record 80e, a manufacturing date record 80f, a sales date record 80g, a last use record 80h, a total usage record 80i, a service status record 80j, an event record 80k, a software and hardware version log 801, a device profile record 80m, and one or more additional data records 80n. Each of these records are populated with information that is received from vendor's enterprise system 24, the medical facilities 26, and/or any computer devices 36 that are separate from system 24 and facilities 26, but in communication with service 22. In combination, the data contained within all of the records 80 constitutes what can be considered a digital replica 46 of the device. That is, the data in records 80 is sufficiently extensive so as to constitute a digital equivalent of the actual physical medical device 32.

Many of the records 80 include multiple pieces of information, rather than just a single piece of information. For example, the device location record 80e includes different levels of location granularity. These include the city, state, address, campus, building, wing, floor, room, bed bay, GPS coordinates, building coordinates, and/or other granular levels of location. System 20 populates these different levels of location as it is able. Some location information it receives, as will be described below, only allows system 20 to determine and/or update one or a subset of these location levels (e.g. some location information may only indicate the room that a medical device 32 is located in), while other location information may allow system 20 to populate all of these different levels of location granularity.

Most of records 80 contain information that is dynamic and changes over time. (Manufacturing date record 80f is one exception). These dynamic records, in addition to storing a current set of data, also store a log of the previous sets of data (if any), and/or changes that were made to the records. The log also records who the individual is or was who changed the record 80 (if applicable) or what event caused the record 80 to be updated or otherwise changed, as well as a time at which the record was changed. System 20 enables an individual with the requisite authorization to perform searching of records 80 based on time of change and/or who or what changed them. The results of such searching are displayable on user interfaces 38 or 39.

In the illustrated embodiment, management service 22 of equipment management system 20 does not receive any Protected Health Information (PHI) from medical device 32 and/or local management server 34 that is subject to the privacy provisions of the United States' Health Insurance Portability and Accountability Act of 1996 (a.k.a. HIPAA). In an alternative embodiment, some of the data sent to management service 22 by the medical devices 32 and/or local management server 34 does include Protected Health Information, and system 20 is modified to ensure that appropriate safeguards are built into the system to ensure compliance with HIPAA. Alternatively, system 20 may be designed to strip away any personally identifiable information in order to transform protected health information into unprotected health information.

In the embodiment shown in FIGS. 1 and 2, the vendor's enterprise system 24 is the enterprise system of the vendor that sells and/or manufactures the medical devices 32. It will be understood by those skilled in the art that, in at least some embodiments, equipment management system 20 is modified to communicate with multiple vendor enterprise systems 24 where each of the systems 24 represents a different manufacturer and/or seller of medical devices 32. In such a modified system 20, the medical devices 32 under the management of system 20 are not necessarily all homogenous in terms of their manufacturer and/or seller. Instead, such a modified system 20 is capable of managing medical devices 32 having a heterogeneous source of manufacturers and/or sellers.

Still further, in some modified embodiments of system 20, a single vendor's enterprise system 24 is included, but the single enterprise system 24 is populated with device data regarding medical device's manufactured and/or sold from third party vendors. In this manner, the modified system 20 is able to manage medical devices 32 having heterogeneous manufacturers and/or sellers without necessarily communicating with multiple enterprise systems 24 of different vendors.

Medical Devices 32

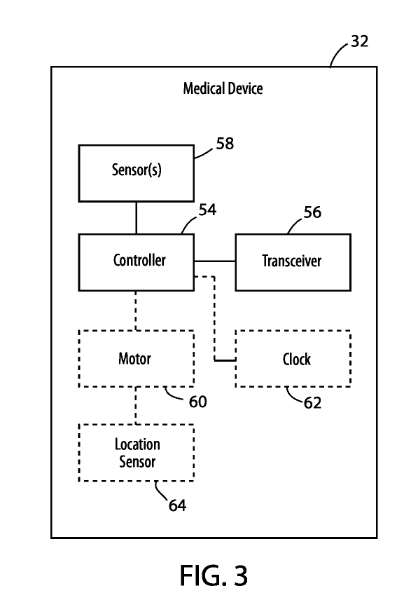

FIG. 3 illustrates an exemplary medical device 32 for use with equipment management system 20. As shown therein, medical device 32 includes a controller 54, a transceiver 56, and one or more sensors 58. In addition, depending upon the specific type of device it is, medical device 32 may also include any one or more of the following: a motor 60, a clock 62, and/or a location sensor 64. Each of the components of medical device 32 shown in FIG. 3 may vary from medical device 32 to medical device 32. However, each of these components shares a set of general characteristics that is described in more detail below.

The controllers 54 include one or more microcontrollers, microprocessors, and/or other programmable electronics that are programmed to carry out the functions described herein. It will be understood that the controllers 54 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions on an individual medical device 32, or they may reside in a common location on the medical device 32. When physically distributed, the components may communicate using any suitable serial or parallel communication protocol, such as, but not limited to, CAN, LIN, Firewire, I-squared-C, RS-232, RS-485, etc.

Transceivers 56 allow the medical devices 32 to communicate with any one or more of the following entities: a local network appliance 30; a localized gateway 30a; a local management server 34; another medical device 32; and/or management server 44 (which may be done by an open port (e.g. HTTP port 80 or HTTPS port 443), or via a suitable tunneling agent, or other structure, that enables the medical device 32 to tunnel through the medical facility's firewall for direct Internet communication, or which may be done indirectly). In some medical devices 32, multiple transceivers 56 are included that utilize different protocols and/or communication media. Transceivers 56 include any one or more of the following: a WiFi radio; a Bluetooth radio; a Zigbee radio; a wired Ethernet port; a serial port (e.g. a Universal Serial Bus (USB) port); an infrared transceiver; and/or a near field communication (NFC) transceiver.

Sensors 58 measure one or more characteristics of the medical device 32 and vary depending upon the particular type of medical device 32. For example, in a patient support apparatus, such as a bed, sensors 58 may include any one or more of the following: an electrical current sensor for measuring electrical current delivered to various motors 60; one or more load cells for measuring the weight supported on the patient support apparatus; one or more force sensors for measuring an amount of force applied to the patient support apparatus, particularly for patient support apparatuses that have built-in propulsion systems; one or more temperature sensors for measuring the temperature of a component of the patient support apparatus; one or more vital sign detectors for detecting a vital sign of a patient supported on the patient support apparatus; one or more pressure sensors for detecting the fluid pressure in a hydraulic lift system; and one or more sensors for detecting when a patient is turned and/or exits from the patient support apparatus.

Multiple examples of the types of sensors 58 that may be incorporated into medical device 32 when medical device 32 corresponds to a bed are disclosed in commonly assigned, U.S. Pat. No. 7,690,059 issued to Lemire et al., and entitled HOSPITAL BED, and commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are hereby incorporated herein by reference. When implemented as a bed, medical devices 32 may also include any of the sensors disclosed in the commercially available S3 bed sold by Stryker Corporation of Kalamazoo, Mich., and documented in the Stryker Maintenance Manual for Stryker's MedSurg Bed, Model 3002 S3, (doc. 3006-109-002 Rev D), published in 2010, the complete disclosure of which is also hereby incorporated herein by reference. Still other sensor combinations may be incorporated into any beds that are part of the managed medical devices 32 of system 20.

Although not shown in FIG. 3, each medical device 32 also includes a memory that contains instructions executed by controller 54. In addition to these instructions, each medical device 32 contains one or more unique device identifiers that are unique to that particular medical device. The unique identifier(s) are stored in a memory accessible to controller 54 and transmitted with the device data that controller 54 sends via transceiver 56. The recipient of the device data therefore knows the specific device to which the transmitted device data corresponds. The unique identifier(s) may be entered by programming the particular medical device 32, by using scanning technology that assigns a unique identifier to a bar code, or other type of code, permanently affixed to the medical device, and/or by other means.

In some cases, only a single unique identifier is stored in the memory of a particular medical device 32. In other instances, multiple unique identifiers are stored in the memory of the particular medical device 32 and the different unique identifiers correspond to different domains of uniqueness. For example, one of the unique identifiers may be an identifier that is unique to the company or entity that manufactured the device. In such cases, the unique identifier distinguishes the particular medical device from all other medical devices made by that same company or entity, but may not uniquely distinguish the medical device from the medical devices may by a different company or entity (who may have adopted a similar or overlapping identification system). A separate unique identifier is included in some embodiments that is unique to not only the company or entity that made the medical device 32, but also to all (or a set of) other manufacturers of medical devices 32. This separate unique identifier is able to distinguish the medical device 32 from a larger population of medical devices 32 than an identifier that is unique only to a single manufacturer or entity. When multiple unique identifiers are stored on a particular medical device 32, the medical device 32 transmits all of the unique identifiers to management service 22, unless otherwise configured by a user or management service 22 to transmit only one (or a subset) of the unique identifiers.

In some medical devices, the unique identifier(s) is or are built into the medical device during its manufacture. In other medical devices, the unique identifier(s) are added after manufacture. Regardless of when the unique identifier(s) are input into the medical device, vendor's enterprise system 24 also includes the unique identifiers, as well as additional information associated with each unique identifier. Such additional information includes, but is not limited to, a human-readable description of the medical device (e.g. a bed, a drill, a pump, etc.); a model, brand, and/or type of the medical device; the date and place of manufacture of the medical device 32; the hardware and/or software versions installed on the medical device 32; options built into the medical device 32; and/or still other information about the device. This information is forwarded by vendor enterprise system 24 to management service 22, which inputs the data into appropriate records 80 within the devices' digital replicas 46 that are maintained for each medical device 32, as will be discussed in greater detail below. The digital replicas 46 also contain the unique device ID (stored in record 80a) and are updated with device data sent from the individual medical devices 32. The data within the digital replicas 46 is made available for display, after suitable processing, on one or more local user interfaces 38 of computer devices 36 (or operator user interfaces 39 of management service 22) that are being used by individuals having the requisite level of access to such information.

In addition to one or more unique identifiers, many of the medical devices 32 are also equipped with software or firmware that enables the medical device to forward device data to management service 22. Such medical devices 32 include a cloud connectivity adapter, which comprises software and/or hardware enabling the medical device 32 to communicate with management service 22 without having to configure each individual medical device 32 to enable such communication. The cloud connectivity adapter, which may utilize conventional technology—such as, but not limited to, the Axeda Connect technology marketed by Axeda Corporation of Foxboro, Massachusetts—enables the medical devices 32 to tunnel through the firewall of LAN 28 of the medical facility 26 and communicate with management service 22. Other technology can, of course, be used.

Regardless of the specific technology used, each medical device 32 includes address data contained within it identifying the address or addresses of the entities it will communicate its device data to. For some medical devices, this address data merely comprises the IP address of management service 22. For other medical devices, this address data includes a local address for local management server 34 and no address for management service 22. Some other medical devices may include addresses for both local management server 34 and for management service 22. For those medical devices that do not include an address for management service 22, such medical devices forward their device data to local management server 34, which does contain the IP address for management service 22. Local management server 34, in turn, forwards the device data to management service 22.

As noted previously, in addition to one or more controllers 54, transceivers 56, and sensors 58, any one or more of the medical devices 32 may also include one or more motors 60, one or more clocks 62, and/or one or more location sensors 64. Motors 60 are typically included in a wide variety of medical devices 32, such as surgical hand pieces used for sawing, drilling, and/or other motorized motion, patient support apparatuses, ventilators, respirators, and operating tables. When a particular medical device 32 includes one or motors 60, controller 54 gathers and transmits data about the motor usage, such as current draw, torque, temperature, usage time, etc., as will be discussed in greater detail below.

When a particular medical device 32 includes a clock 62, controller 54 utilizes the clock 62 to time stamp and date stamp events that occur with respect to the medical device 32. Such events include the detection of errors, the starting and stopping of the device (or a component thereof), the movement of the device from one location to another, the changing of one or more settings of the device by a user (e.g. the arming and disarming of an exit detection system on a patient support apparatus), the commencement and/or completion of a maintenance type of task (e.g. the coupling of a medical device to a battery charger, and the completion of the battery recharging process); and the commencement and/or completion of a medical procedure (e.g. the starting and finishing of a surgical procedure). In general, those medical devices 32 having a clock 62 time and date stamp substantially all of the device data generated by the medical device 32 and forwarded to management service 22.

Medical Device Communication

Each medical device 32 is configured to send its device data to management service 22 based on the individual manner in which that particular device, or class of devices, has been configured. That is, communication between management service 22 and each medical device 32 initially takes place solely based upon medical device 32 sending an initial communication to management service 22. In response to receiving a message from a particular medical device 32, management service 22 is able to send data and/or requests back to that particular medical device 32. However, until an individual medical device 32 makes initial contact with management service 22, management service 22 does not have a way to communicate with that particular medical device 32. Medical devices 32 therefore “call” the management service 22, rather than the management service 22 “calling” the medical devices 32.

In some embodiments, medical devices 32 contact management service 22 using a standard Hyper Text Transfer Protocol (HTTP) port, such as port 80, or an HTTP Secure (HTTPS) port, such as port 443. In such embodiments, the use of these standard ports generally enables the medical device 32 to send outgoing messages to management service 22 that are not blocked by the medical facility's firewall. Further, this type of communication generally allows responses from management service 22 to be passed to medical devices 32 without being blocked by the medical facility's firewall. In this manner, the communication between medical devices 32 and management service 22 does not generally need specialized network settings or configurations that require extensive IT time and effort. Instead, communication is initiated automatically once the medical device 32 has established communication with the LAN 28.

Medical devices 32 are not only configured prior to installation at a medical facility 26 to send messages to a particular IP address (that matches management service 22), but they are also configured prior to installation to know what data is to be sent to management service 22 and when (including how often) such data is to be sent. Once communication is initiated by a medical device 32 with management service 22, management service 22 may respond by changing one or more of the initially configured communication parameters for that particular medical device 32. That is, at any time when management service 22 responds to a message from a particular medical device 32, it may send a command changing when or how often data from a medical device 32 should be sent in the future, and/or what type of data should be sent by the medical device 32 in the future.

Medical devices 32 are configured (either initially or by management service 22 after communication is established) to report their data based upon a subscription that is set up between management service 22 and the particular device 32. Initially, medical devices 32 are configured to establish a specific subscription, but management service 22 may change this subscription after communication is established with the medical device 32. Two general types of subscriptions are an “on-time” subscription and an “on-event” subscription. On-time subscriptions are subscriptions wherein the medical device 32 transmits one or more particular items of data every time a particular time period passes (e.g. once every second, once every minute, etc.). On-event subscriptions are subscriptions wherein the medical device 32 transmits one or more particular items of data whenever one or more events occur. For example, if medical device 32 is a bed, a subscription may be set up for that bed 32 to send data to management service 22 every time a patient exits from the bed (as detected by one or more sensors 58 on the bed).

Data reporting by medical devices 32 to management service 22 can also be defined based on other types of subscriptions. As one example, data reporting may be an “on-limit” subscription. In an on-limit subscription, medical device 32 does not transmit one or more items of data to management service 22 until a threshold has been reached or exceeded. For example, if a bed includes a scale system, the bed may be configured to transmit data to management service 22 regarding the amount of electrical current drawn by a particular motor on the bed only if the amount of electrical current exceeds a particular threshold. The on-limit subscription may also be configured to have a device 32 report its data to management service 22 only if a particular data item crosses the threshold in a particular direction. For example, a bed might be configured to send weight data to management service 22 if an excessive weight is applied to the bed (e.g. over 500 pounds), but not send data to management service 22 when the excessive weight is removed. Hysteresis may also be incorporated into the data reporting such that the timing of when data is reported by the medical device 32 is based upon when the data was previously reported.

Data reporting may also be controlled based upon combinations of any of the aforementioned techniques. As one example, a combination of both on-time and on-event reporting may occur when a particular threshold is crossed and the threshold remains crossed for more than a predetermined time. For example, if a charge on a battery is depleted below a threshold value, the battery (or a charger coupled to the battery) may report data regarding this event to management service 22. If the battery remains below this threshold for more than a specified amount of time (particularly if the charger is attempting to recharge the battery and its charge level is not increasing at an expected level), the battery (or charger) may be configured to report this event. As another example, data may be reported based upon a change that occurs either within or outside of a range. That is, change in one or more pieces of data are not reported every time they change, but instead are reported only when such changes cause the data to fall outside of, or in some cases inside of, one or more defined data ranges.

Still other combinations of when to report data may be implemented, including Boolean combinations of any of the conditions described above. For example, a particular medical device 32 may be configured to report data item X if sensor data A crosses threshold B (in any direction) OR sensor data C falls below threshold D. In this example, the terms X, A, B, C, and D are generic variables that may refer to different data items and/or thresholds, depending upon the medical device 32 and its configuration. Still other types of combinations of when and what data to report are possible.

Data may also, or alternatively, be reported by a medical device 32 based upon a specific request made by management service 22 in response to a communication received from a medical device 32. Thus, when management service 22 responds to a message from a particular medical device 32, management service 22 may request that the medical device subsequently report one or more particular data items, regardless of whether or not those particular data items meet any of the other criteria for reporting them. In other words, whatever data reporting configuration is implemented, management service 22 can override that reporting configuration and request data to be reported at times when the reporting configuration would not otherwise send that data.

In some instances, one or more medical devices 32 report data based upon their location. That is, the medical device 32 is either initially configured, or instructed by management service 22 after communication is established therewith, to report data in a manner that is dependent upon the current location of the medical device. As an example, a particular medical device 32 may be configured to report data X when event Y occurs if event Y occurs at location Z, but not if event Y occurs at location W. This, of course, is merely one example of the type of manner in which location-based data reporting may be implemented. One or more medical devices 32 may therefore report their data to management service 22 (directly or indirectly) in different manners depending upon their location. Methods by which the location of a medical device 32 is determined are described in more detail below.

Although the communication of data from medical devices 32 to management service 22 has been, and is, primarily described herein with respect to sensor data generated from one or more sensors 58 coupled to the medical device 32, it will be understood that some or of all of the data that is transmitted by a medical device to management service 22 may be data that is derived from one or more sensors 58 or from other sources. That is, medical devices 32 do not necessarily send only sensor 58 data, but may receive data from one or more sensors 58 and/or other sources, process the data in accordance with one or more algorithms stored on the medical device, and then send the result(s) of the processed data to management service 22.

Some medical devices 32 of equipment management system 20 do not communicate directly with management service 22. Instead, such medical devices 32 communicate with one or more proxy devices that then communicate with management service 22. For example, some medical devices 32 do not include the communication technology necessary to communicate over LAN 28 with management service 22, but instead include other types of local communication technologies, such as, but not limited to, a USB cable, an RS-232 cable, another type of serial cable, a Bluetooth transceiver, a ZigBee transceiver, etc. Such devices are configured to transmit their device data to a proxy device, such as, but not limited to, localized gateway 30a, which then processes and/or forwards the data onto management service 22.

As will be discussed more below, batteries are one example of devices that may communicate with management service 22 using a proxy. In some embodiments, batteries communicate their data to a battery charger when they are coupled to the battery charger. The battery charger, in turn, includes the communication hardware and software necessary to forward this battery data to management service 22, along with its own data. Messages received back from management service 22 by the battery charger that are intended for the battery are passed onto the battery by the battery charger when the battery is coupled thereto.

As another example, some devices 32 may communicate with local management service 34, rather than management service 22. For example, in some embodiments, hospital beds 32 may be configured to send their medical data to a particular server on LAN 28, rather than sending their data to an IP address that corresponds to cloud based management service 22. In such instances, the local server (which provides local management service 34) acts as a proxy device for communicating messages and/or data back and forth between the beds and the management service 22. That is, the local management service 34 includes the IP address corresponding to management service 22 and forwards the received bed data to management service 22, either with or without further processing of the bed data.

In still other embodiments, some medical devices may communicate their device data to a first proxy device that then forwards that data to another proxy device. For example, in some instances, a battery powered hand piece may not include the means to communicate with LAN 28. In some such instances, the device communicates its device information to a rechargeable battery in the device. The battery then forwards this data, along with its own data, to the battery charger. The battery charger forwards the device data from the handpiece, the battery, and itself to management service 22. The battery charger therefore acts as a proxy device for communications between management service 22 and both the battery and the handpiece.

When medical devices 32 report data to management service 22, either directly or via one or more proxy devices, the reported data is time stamped by the medical device 32 (if it includes a clock) and/or by the one or more proxy devices (if they include a clock) that are used to communicate the data to the management service 22. In this manner, management service 22 receives not only the device data, bus also an indication of when the data was transmitted. Management service 22 may also time stamp the received data according to its own local clock and, in some cases, compare the difference between the two time stamps. If the difference exceeds a threshold, an alert is generated so that appropriate personnel can investigate the reason for large discrepancies between the time at which data is sent and the time at which it is received. Management service 22 retains the transmitted time and data stamps, as well as its own time and data stamps, in the digital device replicas 46. User interfaces 38 and/or 39 can be used by authorized individuals to search for data according to the times and/or dates at which data was sent and/or received. Equipment management system 20 also uses this time and date information to carry out audits and other forms of processing of the received data.

Device Profile Information

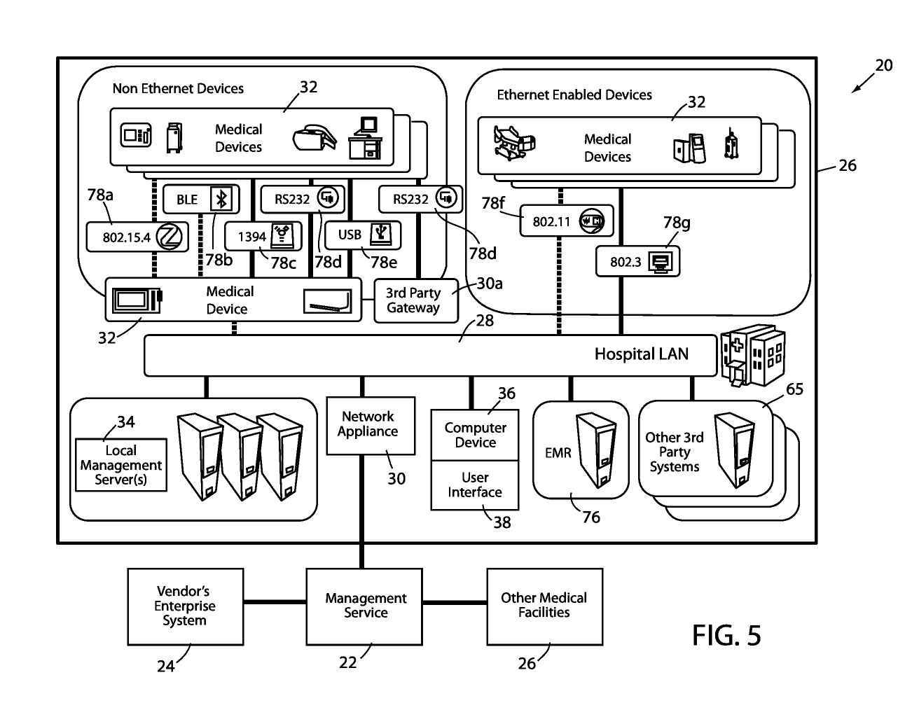

Device profile record 80m of each digital replica 46 (FIG. 5) includes information regarding the communication abilities and configurations for each of the medical devices 32. Management service 22 uses this information in order to manage the information that is exchanged between medical devices 32 and management service 22. For example, device profile record 80m, which can be dynamically changed by the operator of management service 22 using an operator interface 39, includes configuration data indicating what type of data each type of medical device 32 is to send to management service 22, as well as when that information is to be sent (e.g. on-time, on-event, on-threshold, etc.). This communication data overrides whatever communication configuration data the individual medical device 32 may have been initially programmed or otherwise configured with. For example, a piece of operating room equipment 32 may have been initially manufactured and configured to send its data each time it was done being used in a surgical procedure. In such instances, the operating room equipment 32 will initially report its data to management service 22 in accordance with this initially configured schedule. However, once communication is established by the equipment 32 with management service 22, management service 22 may change this schedule and/or the content of the data to be reported. The operator of management service 22 is therefore provided by management system 20 with the flexibility to dynamically adjust the content and scheduling of data that is reported by each medical device 32 to management service 22.

In addition to configuration settings regarding the scheduling and content of data reported by medical devices 32, device profile record 80m also includes data indicating where items of information are to be located for particular types of medical devices 32. That is, the data in any one or more of the data records 80 may be stored in different locations. Device profile record 80m includes data indicating where the data for each record 80 may be found, and this data is updated as additional data and/or records 80 are added to digital replicas 46. For example, the data contained within sales data record 80g may vary depending upon the type of medical device 32. In some instances, medical devices 32 that are of a first type may be manufactured at a first manufacturing facility located in city X of state Y, and that particular manufacturing facility may use a computer system that can be reached at a first IP address, or by using a particular software application, utility, service, or other means. Further, that particular manufacturing facility may store its sales data in a particular format. A second type of medical devices 32, however, may be manufactured at a second manufacturing facility that uses a second computer system that can be reached at a second and different IP address, or by using a different software application, utility, service, or other means. Device profile record 80m stores data indicating the different IP addresses of this sales data and data indicating the format of the stored sales data so that management service 22 can retrieve this sales data and understand its content, regardless of the different locations it may be stored in and regardless of the different manners it may be formatted.

Although the above-example relates to the location and format for retrieving the data associated with sales data record 80g, it will be understood that device profile record 80m may also and/or additionally store the location and format data for each of the other data records 80 associated with a given medical device 32. Further, in some cases, the data for each data record 80 may be stored in a unique location different from the data for each of the other data records 80. In other cases, all of the data records 80 may be stored in a common location and in a common format.

In some embodiments, management service 22 uses the location and format data stored in device profile record 80m to initially replicate and transfer data to a common location. Thus, for example, when a device replica 46 is initially created and contains, for example, no sales data, management service 22 uses the device profile information for that particular device 32 to retrieve a copy of the sales data in record 80g from vendor's enterprise system 24 (for example), and store a copy of that sales data within data repository server 40. In such an instance, there are then multiple instances of the sales data for that particular device. Whenever multiple instances of any particular item of data for medical device 32 exist, device profile record 80m is configured to include logic for determining the source of truth in the case of data discrepancies between the different copies of the supposedly same data, and/or for determining which location to query when multiple copies of data are stored at different locations.

For example, if a first set of sales data corresponding to sales data record 80g is stored at vendor's enterprise system 24 and a second set of the sales data for sales data record 80g is maintained in data repository server 42, device profile record 80m includes data indicating which one of these sources to query first whenever sales data corresponding to a particular medical device 32 is requested by application server 42. Device profile record 80m further includes instructions indicating whether to query the second source of data and, if so, what to do if there is a discrepancy between the data from the first and second sources. These instructions, as well as any and all of the data contained with device profile record 80m can be dynamically adjusted by authorized personnel of the entity operating management service 22 using operating user interfaces 39.

Device profile record 80m also includes instructions regarding where to store data received from medical devices 32 and/or where to store other received data regarding a particular medical device 32. Device profile 80m therefore keeps a dynamically updated map of where each of the records 80 for each digital replica 46 of a medical device 32 is stored, and where updates to those records should be stored. The locations of where all of the data lives for a particular medical device 32 are therefore found in the device profile record 80m corresponding to that particular device 32.

Device profile record 80m is used by management service 22 when a new medical device is detected. That is, when a medical device 32 initially sends data to management service 22, management service 22 checks to see if it has ever received communication from that particular medical device 32 before. It does this by comparing the unique identifier that is contained in the message from the medical device 32 to a database that management service 22 maintains of all of the medical devices 32 that it has previously communicated with. If this check indicates that the medical device 32 has previously sent data, management service 22 retrieves the device profile record 80m for that particular device and uses it to determine where to store the incoming data from that particular medical device 32.

If, however, management service 22 has never previously received data from that particular medical device 32, management service 22 uses the unique identifier contained within the message from that particular medical device 32 to determine what type (and in some cases, what specific model) of medical device the medical device 32 is. Management service 22 accomplishes this by consulting a database stored in data repository server 40 (or elsewhere) that maps the unique device identifiers to their corresponding device types (and/or models). Once management service 22 determines what type (and/or model) of device it has received this initial communication from, it creates a new digital replica 46 corresponding to this particular medical device 32. It creates this new digital replica 46 by first retrieving the data stored in the device profile record 80m for that particular device.

For example, if a new bed is installed at hospital X and sends bed data to management service 22 for the first time, management service uses the unique identifier that is sent with the message containing the bed data to determine that the message came from a bed (the type of medical device), and in some cases, the specific model of bed. Once this is determined, management service 22 retrieves from data repository server 40 (or elsewhere) a device profile record 80m that corresponds to this particular model of bed. As noted, the device profile record 80m includes information indicating where all of the other records that comprise the digital replica 46 of that particular bed can be found. Management service 22 then uses this information from device profile record 80m to determine where to store the incoming bed data for that particular bed, as well as where to retrieve information for that particular bed that corresponds to any of the records 80a through 80l. A unique digital replica 46 of the bed is thus created and, although the contents of the digital replica may be distributed in different locations, the device profile record 80m provides a unified location that defines where the remainder of the digital replica 46 of that bed may be found.

Some medical devices 32 of system 20 include their own integrated location sensor 64. Location sensor 64 may take on a wide variety of forms. In some embodiments of equipment management system 20, one or more of the medical devices 32 include location sensors 64 that include a software module operating in conjunction with one or more of the following components built into the medical device 32: a GPS transceiver, a cellular telephony transceiver, and/or a WiFi transceiver. With any of these types of transceivers, controller 54 uses the outputs from location sensor 64 and known methods of triangulation to determine the location of medical device 32 within a particular medical facility 26.

Equipment management system 20 may also include one or more medical devices 32 having a location sensor 64 that operates as described in commonly assigned U.S. Pat. No. 8,102,254 issued to Becker et al. and entitled LOCATION DETECTION SYSTEM FOR A PATIENT HANDLING DEVICE, the complete disclosure of which is hereby incorporated herein by reference. The location sensors disclosed in this '254 patent include a plurality of fixed location beacons that transmit a unique signal in a relatively localized area. When location sensor 64 detects the unique localized signal, controller 54 concludes that the location of the medical device 32 corresponds to the location of the particular beacon broadcasting that localized signal. The locations of each of the fixed beacons is surveyed during their installation in the medical facility 26 and stored locally on local management server 34, transmitted to one or more of the medical devices 32 via transceiver 56, and/or forwarded to management service 22.

Equipment management system 20 may also operate in conjunction with medical devices 32 that do not include any location sensors built into the medical devices 32. For some of such medical devices 32, system 20 may not be able to determine the specific location of those medical devices 32 within a particular medical facility 26. However, in some instances, the medical facility 26 may include a separate real time locating system (RTLS) that keeps track of the locations of medical devices 32 and shares this location information with system 20. In such systems, medical devices 32 need not include an integrated location sensor 64, but instead may have a physically separate structure (such as an RF ID tag) attached to medical device 32. The sharing of location information between the RTLS system and equipment management system 20 occurs, in one embodiment, by location server 65—which is either a part of, or in communication with, the RTLS system—communicating the locations of medical devices 32 to local management server 34. Other types of information sharing may also occur.

Medical Facilities 26

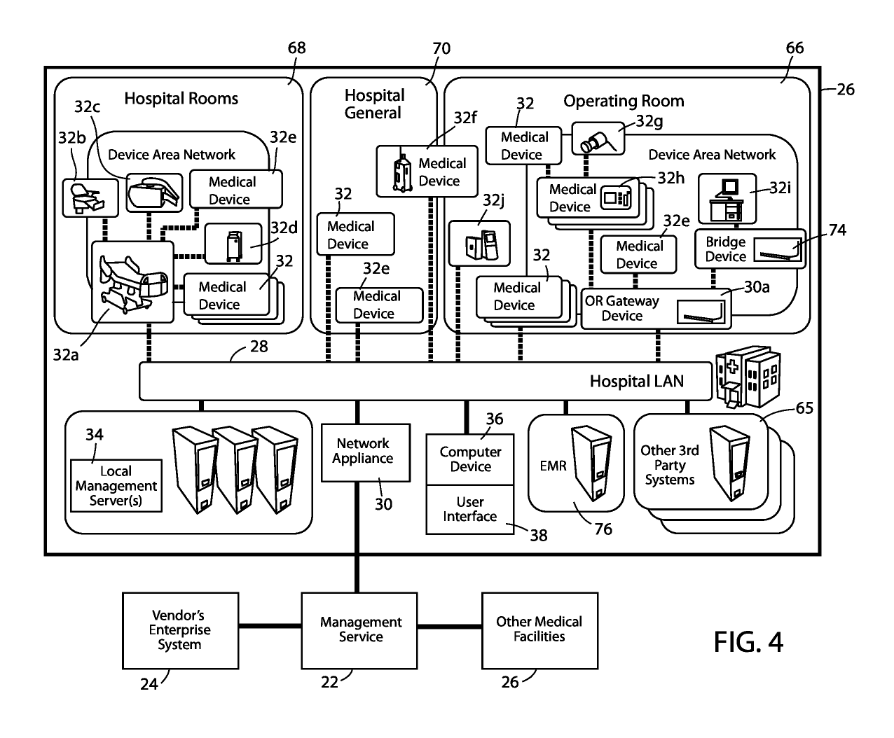

FIG. 4 illustrates in even greater detail one example of the types of medical devices 32 that may be used in a medical facility 26, as well as representative locations of the medical devices 32 within the medical facility 26. The medical facility 26 of FIG. 4 includes one or more operating rooms 66, one or more patient rooms 68, and one or more general areas 70. Medical facility 26 may also include additional areas beyond the three labeled in FIG. 4. However, the three shown in FIG. 4 are representative of typical hospitals and illustrate a basic example of one implementation of equipment management system 20, the principles of which can be applied to a wide variety of medical facilities having different areas and different mixes of medical devices 32.

FIG. 4 illustrates several examples of the types of medical devices 32 that typically may be present in a patient room 68 of medical facility 26. These include a bed 32a, a transport chair 32b, a pump 32c, and a thermal control unit 32d. Additional devices 32 that are of the same type, or of different types, may also be present. Still further, in some embodiments, one or more non-vendor medical devices 32e may be present in patient room 68. Non-vendor medical devices 32e are medical devices that are manufactured or sold by an entity that is not the same as the entity providing management system 20.

Bed 32a, transport chair 32b, pump 32c, and thermal control device 32d may be any of various models that are capable of communicating with LAN 28, either directly or indirectly. One example of such a bed that is suitable for use in system 20 is disclosed in commonly assigned U.S. patent application Ser. No. 14/211,613 filed Mar. 14, 2014 by inventors Michael Hayes et al. and entitled PATIENT SUPPORT APPARATUS WITH REMOTE COMMUNICATIONS, the complete disclosure of which is hereby incorporated herein by reference. One example of a type of thermal control device 32d that is suitable for use in system 20 is disclosed in commonly assigned U.S. patent application Ser. No. 14/282,383 filed May 20, 2014 by inventors Christopher Hopper et al. and entitled THERMAL CONTROL SYSTEM, the complete disclosure of which is also hereby incorporated herein by reference.

Other types of beds 32a and thermal control devices 32d may also or alternatively be used with system 20.

In the example shown in FIG. 4, other than bed 32a, each of the medical devices 32 communicates with LAN 28 using bed 32a as an intermediary. That is, instead of communicating directly with LAN 28, each medical device 32 communicates with bed 32a which relays communications back and forth between the medical devices 32 and LAN 28. The medical devices 32 and 32b-e therefore form a local device area network 72 with bed 32a acting essentially as a router for routing traffic between the devices and LAN 28. It will be understood that the device area network 72 shown in FIG. 4 is but one manner in which medical devices 32 positioned within a patient room 68 are capable of communicating with LAN 28. Any one or more of the medical devices 32 and 32b-d shown in FIG. 4 can be modified to talk directly to LAN 28 without using bed 32a as an intermediary, and/or to be modified to use a different medical device 32 as an intermediary.

FIG. 4 also illustrates a waste management medical device 32f, a non-vendor medical device 32e, and a vendor medical device 32 in general area 70. Waste management medical device 32f may be any waste management system that is capable of communicating with LAN 28, either directly or indirectly. One example of a waste management device 32f that is suitable for use in system 20 is disclosed in commonly assigned U.S. patent publication 2005/0187529 published Aug. 25, 2005 and entitled WASTE COLLECTION UNIT, the complete disclosure of which is hereby incorporated herein by reference. Other types of waste management devices may be used, and waste management devices 32f may be used in other areas of medical facility 26 besides general area 70.

FIG. 4 also illustrates a plurality of medical devices 32 that may be used in operating room 66. These include an endoscopy camera 32g, a power tool console 32h, a device cart 32i, and a surgical sponge counting system 32j. In addition, operating room 66 may contain one or more non-vendor medical devices 32e and one or more other types of medical devices 32. As with patient room 68, one or more of the medical devices 32 within operating room 66 may be in communication with each other by way of a local device area network 72. In the device area network 72 of operating room 66, a localized gateway device 30a acts as a conduit for communications between the medical devices 32 of device area network 72. For example, if endoscopy camera 32g wishes to communicate with device cart 32i (or a device supported thereon), camera 32g sends a message to localized gateway device 30a which forwards the message onto device cart 32i. In this manner, localized gateway device 30a acts in the same manner as bed 32a does of the device area network 72 in patient room 68.

Localized gateway device 30a also connects the medical devices of device area network 72 to the medical facility's LAN 28. In this manner, each of the medical devices 32 of device area network 72 is able to communicate with any of the services, applications, or servers operating on LAN 28. Further, because LAN 28 includes network appliance 30, those devices are able to communicate over the Internet to reach management service 22.

In the example shown in FIG. 4, operating room 66 includes a bridge device 74. Bridge device 74 is a network device that enables medical device 32i to communicate with localized gateway 30a. Bridge device 74 may be a conventional network device that converts the information received from medical device 32i into the communication protocol used by localized gateway device 30a and vice versa.

In some embodiments of equipment management system 20, one or more local management servers 34 are included, such as are shown in FIGS. 4 and 5. In other embodiments, however, it will be understood that local management servers 34 may be omitted. When one or more local management servers 34 are included, the local management server 34 performs one of more of the following functions: (1) receives device data from one or more of the medical devices 32 and forwards the received device data (either modified or unmodified) to management service 22; (2) receives location information from a third party location server 65 indicating the location of one or more of the medical devices 32 and forwards the received location information to management service 22; (3) receives location information from one or more medical devices 32 and forwards the location information to management service 22; (4) determines the location of one or more medical devices 32 using its own internal algorithms and forwards the location information to management service 22; (5) communicates with one or more other servers coupled to LAN 28, such as an electronic medical records (EMR) server 76, and receives additional data from the other server(s) that relates to the medical devices 32; (6) processes some or all of the device data received from medical devices 32; and (7) makes available for display on one or more computers coupled to LAN 28 (e.g. computer devices 36 or servers 65, 76) some or all of the received or processed data from medical devices 32.

Local management server 34 is able to communicate with one or more conventional servers that are installed on LAN 28 by the local IT administrators of the medical facility 26. The specific conventional servers on LAN 28 will vary from medical facility to medical facility. In addition to an EMR or EHR (Electronic Health Records) server, these servers include the following: a financial server that maintains data regarding billing and billing rates; a scheduling server that organizes and maintains scheduling for medical procedures and other patient-related events; an admission, discharge, and tracking (ADT) server that maintains and organizes patient information, including their assigned locations within medical facility 26; a Real Time Location Service (RTLS) server that determines the locations of one or more types of medical devices 32 within the facility 26; a maintenance server that maintains maintenance records, schedules maintenance, and provides alerts or notifications of items needing maintenance; a work flow server that manages the timing of certain events, including, but not limited to, the changing of caregiver shifts and caregiver-patient assignments; and an alerting server that forwards alerts to specific individuals via pagers, texts, emails, cellular phones, beepers, and/or other means.

FIG. 5 illustrates in greater detail an illustrative set of communication protocols that may be incorporated into equipment management system 20 and used for communication between medical devices 32, LAN 28, and local management server(s) 34. It will be understood that the communication protocols shown in FIG. 5 are merely an illustrative example of the variety of communication protocols that may be used with system 20, and that any particular installation of equipment management system 20 may use a different set of these communication protocols, or, in some cases, only a single communication protocol. As can be seen in FIG. 5, any one or more of the medical devices 32 may communicate with LAN 28 using a ZigBee protocol 78a (IEEE 802.15.4), a Bluetooth protocol 78b (IEEE 802.15.1), a Firewire protocol 78c (IEEE 1394), an RS-232 protocol 78d, a serial protocol 78e (such as Universal Serial Bus (USB)), a WiFi protocol 78f (IEEE 802.11), and an Ethernet protocol 78g (IEEE 802.3).

Further, any medical device 32 that is configured to communicate using a specific one of these protocols can communicate with any other medical device 32 that uses the same protocol. As a result, any medical device 32 can be communicatively coupled to another medical device 32 that “speaks” the same protocol and use the other device as a proxy for communicating with management service 22.

The various communications protocols 78 shown in FIG. 5 may be used by any of the medical devices 32 when the devices communicate with another medical device 32, or with a localized gateway 30a, with a bridge device 74, or with a wired or wireless access point of LAN 28. Typically, although not necessary in all cases, the device data is converted to either the Ethernet communication protocol 78g or the WiFi protocol 78f by the time it makes its way onto LAN 28. If necessary, network appliance 30 or another network device converts the device data received in the WiFi protocol 78f or Ethernet protocol 78g into TCP/IP packets for transmission to management service 22. The conversion of device data from any of communication protocols 78a, b, c, d, and/or e to communication protocols 78f or 78g may be done by a medical device 32 having built into protocol conversion technology, by a localized gateway 30a, and/or by a bridge device 74.

Medical devices 32 send their unique identifier as part of their device data that uniquely identifies themselves and distinguishes themselves from all other medical devices 32. In some embodiments, equipment management system 20 receives device data from multiple medical facilities 26. Further, in some of these multi-facility embodiments, local management server 34 and/or medical devices 32 are configured to send location information to management service 22 that indicates what facility the medical devices are located in. Indeed, in some embodiments, local management server 34 and/or medical devices 32 are configured to forward location information of greater granularity than simply the medical facility they are located in (e.g. data indicating in which floor, room, bed bay, exact set of coordinates, and/or other specific locations within medical facility 26 the devices 32 are located).

However, in some multi-facility embodiments, equipment management system 20 does not forward any location information to management service 22. In these embodiments, equipment management system 20 is configured to determine which medical facility the medical devices are located in by consulting the device sales records 48, which may be stored in the vendor's enterprise system 24, management system 22, or elsewhere. For example, a bed 32a may send device data indicating that a patient is currently occupying the bed 32a. The device data identifies the specific bed 32a with its unique identifier(s). The bed's device data, however, does not indicate which patient room 68 the bed 32a is located in. The LAN 28 forwards the device data received from bed 32a to management service 22 using the TCP/IP protocol. The packets sent using the TCP/IP protocol each include a destination IP address corresponding to management service 22 and a source IP address corresponding to the medical facility 26. However, the packets of device data received by management service 22 will not include any location information about bed 32a other than the source IP address of those packets. In some embodiments, management service 22 includes a table of IP addresses and the medical facilities associated with them and uses the table to determine which medical facility bed 32a is located in (or management service 22 connects to a geo-location service that relates IP addresses to geographic locations).

However, in other embodiments, management service 22 utilizes the device sales records 48 to determine which medical facility 26 bed 32a is located in. Management service 22 determines the location of bed 32a using the sales records 48 by searching the sales records 48 for the record corresponding to the unique identifier or identifiers of bed 32a. Once the sales record for bed 32a is identified, management service identifies the customer to which bed 32a was sold (which is stored in the sales data record 80g of digital replica 46) and uses the location associated with that customer as the location of bed 32a. In other words, service 22 assumes that bed 32a is located on the premises of the customer who purchased bed 32a, according to the sales record information stored in sales data record 80g, which may be provided by enterprise system 24 or received from other sources.

When possible, sales records 48 identify not only the customer who purchased the bed 32a, but also the city, state, and address of the purchaser, as well as the city, state, and address of the campus, building, or other structure where the bed 32a is intended to be located (if different from the address of the purchaser). Thus, for example, if a sales record 48 identifies XYZ Corporation as the purchaser, and XYZ Corporation operates fifteen different hospitals in three different states, the sales record 48 identifies, if possible, which of the different hospitals (by address, campus, and/or building name) the bed 32a is going to be used at and/or delivered to.

Once management service 22 has located the sales record 48 for bed 32a, it updates the location record 80e of the digital replica 46 of bed 32a by indicating that the last known location of bed 32a is at the medical facility 26 corresponding to the customer who purchased bed 32a. Management service 22 may also add a time stamp to this location determination. Management service 22 thereafter makes this location information available to computer devices 36 located within a given medical facility, or outside a medical facility. The local user interfaces 38 of such computer devices 36 is therefore able to display the customer's location as the last known location of bed 32a when those local user interfaces display information related to bed 32a.

For some medical devices 32, management service 22 determines the location of the medical device 32 based upon the location of another device that communicates with or couples to the medical device. For example, some medical devices, such as electrically powered surgical hand pieces that operate on a rechargeable battery, communicate their device data to the rechargeable battery while the battery is coupled to the hand piece. The battery stores this hand piece information and generates its own device data about itself. When the battery is removed from the hand piece and placed in a charging station, the battery communicates not only its own battery device data to the charger, but also the hand piece data that the battery received from the hand piece. The charger then forwards the battery device data, the hand piece device data, and its own device data to management service 22.

Certain medical devices 32 are often stationary within a given medical facility, such as, for example, battery charging stations. In some embodiments of equipment management system 20, the locations of such stationary medical devices within medical facility 26 are input into system 20. Inputting this data may be accomplished via one or more controls or ports on the device itself, via a computer device in communication with local management server 34, or via a computer device (e.g. local computer device 36) that communicates with management service 22. When inputting this location information into management service 22, a computer device that is not local to the corresponding medical facility may also be used. Regardless of the path by which the locations of stationary medical devices are input into system 20, management service 22 enters the location information into the location records 80e of the device's digital replica 46 for the corresponding medical devices 32. This location information is then made available by management service 22 for display on any user interfaces that are in communication with management service 22 and that have the requisite access to this location data (e.g. local user interfaces 38 or operator user interfaces 39).

User Interfaces 38 & 39

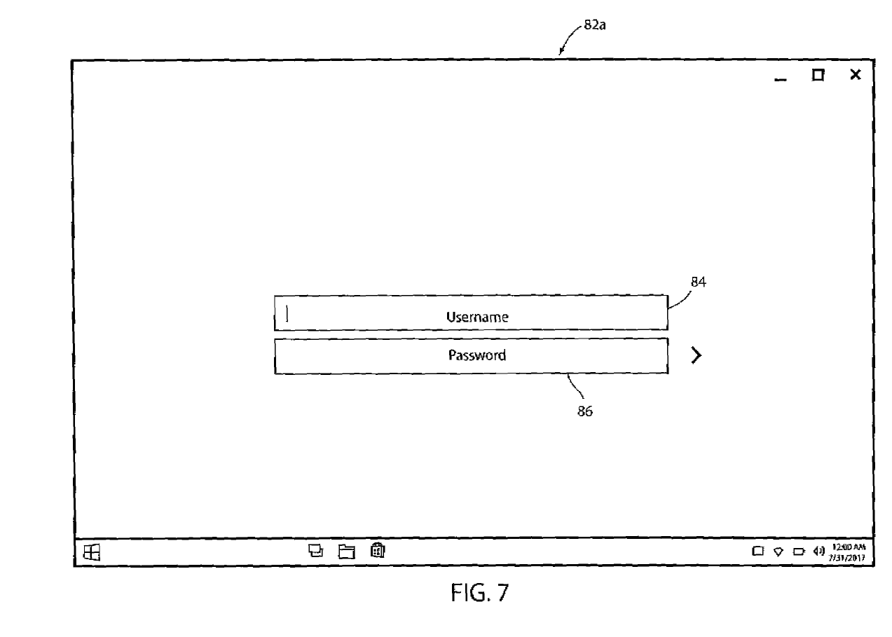

FIGS. 7-21 illustrate a plurality of screen shots 82a-o that constitute a sampling of the types of screen shots that may be displayed on one or more of the local user interfaces 38 of computer devices 36. It will be understood that the data displayed on these screen shots is merely an arbitrary example of the types of data displayable on local user interfaces 38, and that the actual data displayed on user interfaces 38 will vary from system to system 20, depending on the medical devices 32 that are in communication with system 20. It will also be understood that the layout of the information on each of these screen shots 82a-p may be modified from what is shown in these drawings. FIGS. 7-21, however, illustrate a number of the underlying functions of equipment management system 20, which will be discussed in greater detail below.

Still further, it will be understood that any of the data displayed on local user interfaces 38 may also be viewed by appropriately authorized individuals having access to operator user interfaces 39. Thus, the operator of management service 22 can view the device data for each of the medical facility's 26 that subscribe to, or otherwise, use, management service 22. This enables the operator of management service 22 to run reports, view data, verify functionality, and perform other tasks for both the benefit of the user of management service 22 (e.g. one or more medical facilities 26) and for the efficient maintenance, operation, and/or upgrading of management service 22. It will therefore be understood that, although the following description of the types of screen shots that are displayable by management service 22 are discussed with respect to user interface 38, the following discussion is equally applicable to operator user interfaces 39.

The underlying functionality associated with screen shots of FIGS. 7-21 may be carried out solely by the computer device 36 associated with the local user interface 38 which is displaying the screen shot, or it may be carried out solely by application server 42, which is in communication with the computer device 36, or it may be carried out by a combination of both local program or script execution on computer device 36 and remote program execution on remote application server 42. It will be understood that the much of the data displayed on the screen shots of FIGS. 7-21 is generated by remote application server 42 is gathered from its communication with data repository server 40 and other servers.

As was noted previously, user interfaces 38 may take on a variety of different forms within a given embodiment of equipment management system 20. Some user interfaces will consist of a conventional desktop computer display coupled to one or more input/output devices, such as, but not limited to, a keyboard and/or a computer mouse. Some user interfaces 38 may consist of a touchscreen computer display in which the user touches the display screen in order to manipulate and control the data displayed thereon and the functionality of computer device 36. Other user interfaces 38 may vary further and may be dependent upon the form of computer device 36.

Computer devices 36 may also take on a variety of different forms within a given embodiment of equipment management system 20. One or more of computer devices 36 may be a conventional desktop computer, a laptop computer, a notebook computer, a tablet computer, a Computer on Wheels, a smart phone, or any other type of computing device capable of displaying data of the type shown in FIGS. 7-21 and interacting with application server 42.

FIG. 7 illustrates an illustrative screen shot 82a that is displayed by any of computer devices 36. As was noted previously, a user of system 20 navigates to screen shot 82a by using a conventional web browser to access a web-based system associated with management system 20. When presented with screen shot 82a, the user must enter a user name into an identification field 84 and a password into a password field 86. Failure to present a valid user name and password prevents the user from accessing and utilizing equipment management system 20.

In general, system 20 is set up such that the operator of management service 22 controls which data, features, and functions are available to a particular medical facility 26. Within that set of data, features, and functions, the operator of management service 22 delegates authority to one or more selected individuals, or one or more selected classes of individuals, to decide who within that medical facility 26 will have access to that set of data, features, and functions, and what level of access they will be given to that set of data, features, and functions. In other words, the operator of management service 22 allows the delegated representative of the medical facilities 26 to decide which, and how many, user classes the medical facility wants to utilize, and what type of access to system 20 each class will be allowed. As one example, a medical facility might set up the following classes of individuals: nurses, doctors, technicians, biomeds, janitorial staff, administrators, IT personnel, etc. For each class, individuals within that class are allowed to access certain features and data of the management service 22.

After the medical facility has decided upon the classes of users, it assigns its employees to one or more of the classes and stores this information in a database accessible to system 20 (e.g. local management servers 34, or elsewhere). The medical facility 26 also assigns initial user names and passwords to each of these individuals at the time system 20 is installed in a medical facility 26, as well as when new employees or other authorized personnel are added. These may be changed by the individuals after they log into the system for the first time.

The administrators of the medical facility 26 forward the assigned user names and passwords to management service 22, which then forwards the data to one or more of the application servers 42 which control the communications between management service 22 and computer devices 36. Application servers 42 consult this information when a user attempts to log into system 20 and denies access if the attempted user does not present a valid password and user name. Application servers 42 also use this information to determine what information, features, and functions are available to a particular user who does present a valid password and user name. This determination is based upon the class to which that particular individual has been assigned.

In addition to users who are associated with one or more medical facilities 26, system 20 may also be used by other individuals who are not associated with a medical facility 26. Such users include individuals associated with a vendor's enterprise system 24, individuals associated with service centers for servicing medical devices 32, sales personnel who sell one or more medical devices 32, and still other individuals. An operator of management system 20 may use one of the operator user interfaces 39 to enter user names and passwords of such individuals who are not associated with a particular medical facility 26. As noted, such individuals may include sales personnel, technicians, repair personnel, and/or people associated with the operation of management service 22, and/or enterprise system 24.

In addition to the user names and passwords of individuals not associated with a medical facility 26 (i.e. those whose access privileges are not determined by a medical facility administrator), the administrator of equipment management system 20 may also assign access privileges to such users. Such access privileges are based upon one or more classes that the administrator of system 20 has defined. The classes may overlap with one or more of the classes used by the administrators of medical facilities 26, or they may be different. Examples of such classes include: technicians, IT personnel, service personnel, sale personnel, engineers, etc. System 20 automatically limits the data that can be displayed to a particular user of system 20 based upon the class of that individual. Thus, for example, a sales agent may be able to access the sales price of a medical device and/or the identity of the customer of that device, while a technician may not be able to access that particular data.

The user names and passwords of individuals associated with a particular medical facility are marked in system 20 as being associated with that particular medical facility. That is, when authorized user names and passwords are input into system 20, the particular medical facility (or facilities) to which those individuals are associated, are input into system 20. System 20 uses this information to automatically limit the access those individuals have to the subset of data and functions associated with service 22 for that particular medical facility. Thus, for example, an employee of hospital A is not allowed to view the medical device data from medical devices 32 that are located in hospital B (where hospital B is an independent entity and/or has otherwise not agreed to share its data with personnel from hospital A).

In response to a user entering his or her username and password in fields 84 and 86 of screen shot 82a (FIG. 7), application server 42 compares the entered data with the stored usernames and passwords to determine if valid information has been entered. If not, the user is not allowed access to management service 22. If the entered data does match a stored username and password, the user is allowed to access the device data that is associated with his or her role.

In an alternative embodiment, other types of authentication may be required before a user is allowed to use system 20. Such other types of authentication include, but are not limited to, scanning a badge or other identification card through a card reader or bar code reader, inputting biometric information of an individual, or other taking other steps to establish that an individual has the proper credentials to use system 20.

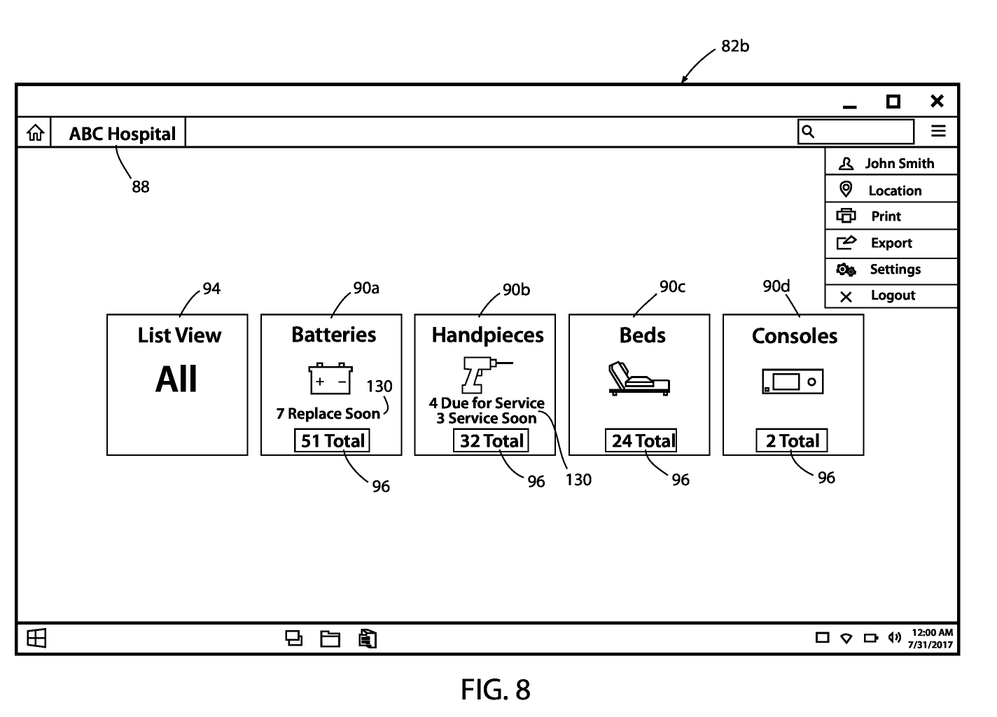

FIG. 8 illustrates a screen shot 82b that is displayable on user interface 38 after an authorized user has gained access to system 20 (i.e. entered the appropriate user name and password in the fields 84 and 86). Screen shot 82b provides an overview of all of the medical devices 32 at a particular medical facility 26 according to different categories of those medical devices. More specifically, screen shot 82b includes a medical facility indicator 88 that identifies which medical facility 26 the devices are located in. The facility identified in indicator 88 of FIG. 8 is ABC Hospital. Screen shot 82b also displays information about the different categories of medical devices 32 that are located at the medical facility identified by indicator 88. Based on information received from management service 22, computer device 36 displays one or more categories 90 of the medical devices 32 located at ABC Hospital. In the screen shot of FIG. 8, four different categories 90 are displayed: a battery category 90a, a hand piece category 90b, a bed category 90c, and a console category 90d. Each of the categories 90 includes a total number indicator 96 indicating the total number of devices at the medical facility 26 (ABC Hospital in this example) that fall within that category, as well as a service summary field 130.

The categories 90 displayed on user interface 38 in screen shot 82b are based upon the medical devices 32 that are currently determined to be at ABC Hospital. That is, system 20 dynamically adjusts the content of screen shot 82b based upon the medical devices 32 that are at a particular medical facility 26. If a particular medical facility 26 has only one category 90 of devices 32 that are present at the facility, then screen shots 82b will only display one category 90. If a particular medical facility 26 has more than the four categories shown in FIG. 8, of a different set of categories, system 20 will display screen shot 82b in a different way that matches the medical devices of that particular medical facility 26.

In the example of FIG. 8, ABC Hospital includes fifty-one batteries, thirty-two hand pieces, twenty-four beds, and two consoles. The information displayed in screen shot 82b listing the different types of medical devices 32 at ABC Hospital is gathered from data maintained in data repository server 40 and forwarded to application server 42. Data repository server 40, in turn, receives this data from multiple different sources.

For example, data repository server 40 receives data about the location of the medical devices listed in FIG. 8 from the device sales records 48 supplied from vendor's enterprise system 24 or from other sources. As was discussed previously, device sales records 48 identify the customers to which the medical devices 32 were sold, as well as the date they were sold. This information is forwarded to data repository server 40. The corresponding device's digital replicas 46 are updated to indicate that this data is stored in data repository server 40. More specifically, in at least one embodiment, the device's digital replica 46 is updated by updating the device profile record 80m such that the location record 80e in the digital replica 46 points to data repository server 40 as containing the last known or current location of that particular medical device 32. Whenever application server 42 requests a listing of all medical devices located at a particular medical facility 26, data repository server 40 searches the location records 80e for those that correspond to the medical facility from which the request originated.

Thus, for example, in order to generate the data displayed in the example of FIG. 8, data repository server 40 searches the location records 80e for those having ABC Hospital therein. The set of digital replicas 46 that are responsive to this search are then further examined to determine what the types are of each of the devices that are located at ABC Hospital. In order to do this, the devices' digital replicas 46 are consulted in order to determine the locations of the device type records 80b corresponding to the devices 32 at ABC Hospital. Once those locations are determined, data repository server 40 searches the device type records 80b of those devices at Hospital ABC. The results of this search are then forwarded to application server 42, which forwards them to computer device 36 for display on its user interface 38.

Data repository server 40 updates the location record 80e within a given device digital replica 46 whenever it receives updated location information, either from vendor enterprise system 24 or from a medical facility 26. In some embodiments, vendor enterprise system 24 also includes servicing records indicating when a medical device 32 was removed from a medical facility 26 for servicing or maintenance. In those cases, vendor's enterprise system 24 forwards information to data repository server 40 indicating any one or more of the following: an identification of which devices have been taken out for maintenance work, the date of such maintenance work, the location of the maintenance or repair center, the type of maintenance work being performed, the expected date of completion of the work, and the actual date when the work is completed and the medical device is returned to medical facility 26, to the extent these various items of information are entered into vendor's enterprise system 24. Management service 22 then enters this information into the appropriate records 80 (e.g. service status record 80j) of the devices' digital replicas 46 for the corresponding medical devices 32.

System 20 classifies, in some embodiments, the maintenance work into different categories. For example, two maintenance categories that are used in some embodiments include on-site maintenance and off-site maintenance. Some medical devices 32 are too large to be transported from a medical facility 26 to an off-site service center, and therefore are primarily serviced at the medical facility 26 (although not necessarily in the same location(s) in the medical facility 26 that the devices 32 are normally used). When such large devices 32 are taken out of service for maintenance work, system 20 classifies these as undergoing on-site maintenance. Other devices 32, which are typically smaller, are shipped off-site when maintenance work is performed on them. System 20 classifies these as undergoing off-site maintenance when taken out of service for maintenance work.

In some cases, data repository server 40 also updates the location record 80e within the digital replicas 46 when it receives device data from a medical facility 26. Depending upon the particular medical device 32, the particular manner in which system 20 is integrated into the medical facility 26, as well as the presence or absence of a location server 65 within a given medical facility (or outside of, but accessible to, that given facility), the device location information forwarded to data repository server 40 will vary in terms of the granularity of the location, the path by which the location information is provided, and the frequency in which the location information is provided.

For example, some medical devices 32 may include one or more sensors that detect their location within medical facility 26. Such medical devices 32 forward this detected location with the device data messages they send to device management server 44, which in turn forwards this information to data repository server 40. When data repository server 40 receives this information, it updates the location record 80e of the corresponding device's digital replica 46 with the received location. It also updates the location record 80e with the time at which the medical device 32 determined its location.

In some instances where medical devices 32 include suitable technology for detecting their own location within a medical facility 26, the medical device 32 may also include suitable technology to detect which particular medical facility 26 it is located in. However, in some instances, the medical devices 32 may only be able to determine a particularized location within a medical facility 26, but not be able to identify the medical facility itself. In these latter instances, the medical devices 32 transmit their particularized location information to management service 22 and data repository server 40 combines this particularized location information with the location information received from device sales records 48. Thus, for example, data repository server 40 may receive device data indicating a particular medical device is located in bed bay A of room 101 and sales record data indicating that that particular medical device was sold to ABC Hospital. Data repository server 40 combines this information into the location record 80e of the corresponding device's digital replica 46 so that the device's digital replica 46 includes data indicating not only what medical facility 26 the medical device 32 is located in, but also a specific location of that medical device 32 with the known medical facility 26.

Other medical devices 32 may not include suitable sensing technology for determining their own location within medical facility 26. However, in some medical facilities 26, other structures may be present that enable location information for the medical devices 32 to be forwarded to management service 22. For example, as mentioned previously, a particular medical facility 26 may use a location server 65 that determines the location of medical devices 32 within that particular medical facility 26. In such cases, location server 65 is configured during the installation of system 20 to either forward this location information directly to management service 22, or to pass this information local management server 34, which in turn forwards the location information to management service 22. Regardless of the source of the location information, data repository server 40 updates the location record 80e with the received location information in the digital replicas 46 of each of the corresponding medical devices 32.

Still other medical devices 32 may have their location input into them after they are installed in a particular medical facility 26. For example, some medical devices 32 may be affixed to walls, floors, or ceilings, or may be simply too bulky to move. In some instances, the location of these devices 32 is input into the device 32 itself using a user interface on the medical device. The medical device 32 then shares this information with system 20 when it communicates with management service 22. In other instances, the location of these fixed medical devices 32 is input into system 20 via a user interface 38 or 39, and management system 20 stores this information in location record 80e.

Some medical devices 32 may not be configured to transmit their device data to management service 22 directly, but instead are configured to forward their device data to local management server 34. In those cases, local management server 34 is configured by technicians during the installation of system 20 within a given medical facility to append a location identifier to the device data it receives from medical device 32 and forwards to management service 22. This appended location information identifies the specific medical facility in which local management server 34 is installed and supplements whatever other additional location information that might be forwarded to management service 22.

Still further, data repository server 40 may also use the IP address from network appliance 30, or some other identifier associated with network appliance 30, to determine what medical facility 26 the set of medical devices 32 are located in that use local network appliance 30 to transmit their device data to management service 22.

Regardless of the source, manner, frequency, and/or type of location information that is forwarded to data repository server 40, data repository server 40 maintains a history of all of the location information that it receives so that authorized users can see the complete history of the location of the corresponding medical devices 32.

In some instances, equipment management system 20 may receive conflicting location information from multiple sources. For example, sales records may indicate a particular medical device 32 was sold to hospital XYZ while location sensors within that medical device 32 (or an RTLS system, or another location technology) may report to equipment management system 20 that that particular medical device 32 is located at hospital ABC. Other types of conflicting location information may also occur.

Equipment management system 20 is configured to resolve discrepant location information for medical devices 32 by assigning a trust level to each of the different sources of location information. If two sources of location conflict, system 20 uses the location information from the source with a higher trust level. If more than two sources of location information conflict, combinational logic is used to determine which of the multiple sources of location to use assign an actual location of the medical device 32. In some instances, system 20 looks for transition events with respect to location. Transition events refer to events when the location of a medical device 32 may have, or did in fact, change. By analyzing past location data and other data from a medical device 32, system 20 automatically and autonomously determines if the likelihood of a medical device 32 having changed locations in the past exceeds one or more thresholds. Using this logic, system 20 is able to determine if any of the sources of conflicting location information refer to location information that is out-of-date, or that otherwise doesn't reflect the change in location. In such instances, the more up-to-date location information is used for assigning a current location to the medical device 32. In still other instances, system 20 is configured to flag certain instances of discrepant location information and request a user to confirm and/or assign a location to a medical device 32.

Equipment management system 20 also records in the location record 80e of the digital replicas 46 the source of the location information. In this manner, equipment management system 20 is able to determine if any discrepancies exist between the medical device 32 that are reporting device data from a particular facility 26 and the medical devices that were sold to that facility. For example, equipment management system 20 periodically compares a list of all of the medical devices 32 at a particular medical facility 26 that it has received device data from to a list of all of the medical devices 32 that were sold to that medical facility 26y (this latter information is stored as one component of the data stored in sales data record 80g). If equipment management system 20 detects that it has not received any device data for a threshold amount of time from that medical facility 26 for a particular medical device that was sold to that facility, then it notifies an administrator at that facility that it may have lost or misplaced the purchased piece of equipment. Conversely, if equipment management system 20 receives device data from a medical device 32 that is located at a facility that does not correspond to the facility identified in the sales data record 80g, the system 20 is configured to send an inquiry to the medical facility 26 that originally purchased the medical device asking for confirmation that they have sold or transferred the device. Alternatively, or additionally, system 20 may conclude that the medical device 32 has been properly sold to the other medical facility and may therefore update the sales data record 80g appropriately, as well as the location record 80e.

Returning to FIG. 8, after a user logs into system 20 (e.g. successfully enters an authorized user name and password on screen shot 82a of FIG. 7), and views a screen shot of the type shown in FIG. 8 listing the categories of medical devices 32 currently located at the medical facility 26, the user is able to select any of the various medical device categories (e.g. 90a, 90b, 90c, 90d) to view further information about the medical devices 32 within that category. The user is also able to select an “all devices” icon 94 to view additional information about all of the devices at medical facility 26. The selecting of a specific category 90 or icon 94 is carried out in one or more conventional manners; such as by physically touching the display at the location corresponding to the selected category 90 or icon 94 (for user interfaces 38 having touch screen displays); by using a computer mouse to position a pointer over the selected category 90 or icon 94 and clicking the computer mouse; by using a keyboard to select the category 90 or icon 94; or by other means. Once the desired category is selected, computer device 36 will display on user interface 38 additional information about the selected category 90, or about all of the medical devices 32 if icon 94 is selected. This additional information is supplied to computer device 36 from management service 22.

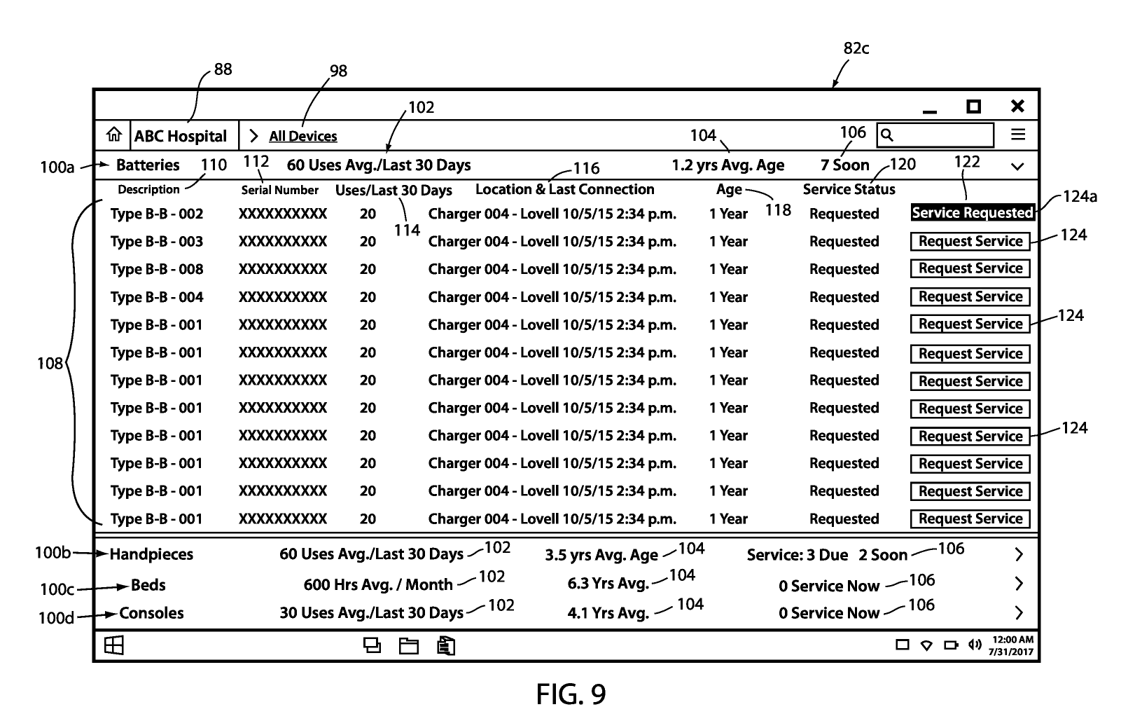

FIG. 9 illustrates a screen shot 82c that is representative of the type and layout of information that may be displayed in response to a user selecting the “all devices” icon 94 of FIG. 8. Screen shot 82c includes medical facility indicator 88, a heading identifier 98, a plurality of category summary bars 100a, b, c, and d. Medical facility indicator 88 identifies the particular medical facility 26 which the data displayed on screen shot 82c corresponds to. Heading identifier 98 provides a summary description of the data that is being displayed in screen shot 82c. In the example of FIG. 9, the heading identifier 98 indicates that the data below it is for all of the devices located at the ABC Hospital.

Category summary bars 100a, 100b, 100c, and 100d correspond to categories 90a, 90b, 90c, and 90d, respectively. Category summary bars 100a-d are viewable in both an expanded form and a contracted form. In the example of FIG. 9, category bar 100a, which corresponds to the battery category 90a, is shown in the expanded form, while category bars 100b-d are shown in their contracted form. Regardless of whether the category summary bars 100 are shown in their contracted or expanded form, each category summary bar 100 includes an average usage field 102, an average age field 104, and a service summary field 106.

The average usage field 102 displays the average amount of use of all of the medical devices 32 within the category 90 identified in category bar 102 whose contents have been expanded and are displayed. For example, category summary bar 100a of FIG. 9 corresponds to the battery category 90a. Average usage field 102 of category summary bar 100a therefore displays the average number of uses of the batteries at ABC Hospital over the indicated time period (in this case 30 days). Similarly, average usage fields 102 of category summary bar 100b, 100c, and 100d of FIG. 9 display the average number of uses of the hand pieces, beds, and consoles, respectively, at ABC Hospital over the indicated time periods.

Average age field 104 displays the average age of the medical devices 32 within the category 90 of medical devices 32 corresponding to that particular category summary bar 100.

Service summary field 106 displays the number of medical devices 32 within the corresponding category 90 that fall within different service categories, such as service due soon or service due now. The “due soon” category corresponds to medical devices 32 that will need servicing within a predefined time window. The predefined time window may vary for individual medical devices 32 such that those medical devices 32 requiring more planning or down time for servicing are flagged sooner. In some embodiments, the predefined time window is user-customizable during the installation of equipment management system 20 so that the administrators of medical facility 26 can choose how soon in advance they receive notification of upcoming service dates. In other embodiments, the amount of advance notification of upcoming service events is fixed.

The “due now” service category refers to those medical devices 32 whose servicing date has passed. In some embodiments, system 20 is configured to display the number of “due now” medical devices 32 in service summary 106 in a different color from the number of medical devices 32 that fall into the “due soon” category. In one such embodiment, system 20 displays the number of “due now” medical devices in service summary field 106 in red and the number of “due soon” medical devices in service summary field 106 in yellow.

The service schedule for each of the medical devices 32 is generated in a number of different manners. In a first manner, the service schedule is set by manufacturer of medical device 32 and stored within vendor enterprise system 24, which communicates the schedule to data repository server 40. In a second manner, the service schedule is set by the administrators of the medical facility 26 and stored in local management server 34, which may forward the service schedule to management service 22. In yet a third manner, some aspects of the service schedule come from vendor enterprise system 24 and other aspects come from local management server 34.

Regardless of the source of the servicing schedule, the servicing schedule may be time based, usage based, or a combination of both. When time based, servicing dates are scheduled based upon how much time has elapsed since a particular medical device 32 was purchased or last serviced. When usage based, servicing dates are scheduled based upon how much usage a particular medical device 32 has undergone since it was purchased or last serviced.

The manner in which a medical device's usage is measured may vary from medical device to medical device. For some medical devices, the amount of time the device is turned on is used to quantify the device's usage. For other medical devices, the number of times the device is activated, or one or more components of the device are activated, is used to quantify the device's usage. Still other medical devices may measure usage based on the number of times the device was used in surgery or another medical procedure, how many patients the devices has been used with, how many times the device has been sterilized or cleaned, and/or how many times the device has been electrically recharged.

In at least one embodiment, management service 22 is adapted to modify and/or supplement the service schedule based upon analyses of the errors, usage, and other data collected by management service 22 from a plurality of similar medical devices 32 gathered over a statistically significant time period. The collected data, in at least one embodiment, is collected from a plurality of different medical facilities 26 and is based upon errors, servicing requests, reported malfunctions, or other data indicating performance issues with one or more medical devices 32. As one example, management service 22 analyzes this data to determine if a statistically significant number of medical facilities 26 have had a particular a problem with a specific type of bed after a specific amount of usage. If that specific amount of usage is less than the scheduled amount of usage between regularly scheduling servicing dates for those beds, then management service 22 alters the scheduled service dates for those types of beds so that they can be proactively serviced prior to them experiencing the particular problem.

In the example shown in FIG. 9, a user is able to click on, or otherwise select, any of category summary bars 100b-d in order to change them to their expanded display form. As can be seen from the expanded display form of category summary bar 100a, expanding a particular category bar 100 causes user interface 38 to display underneath the category bar 100 a listing 108 of all of the medical devices 32 within that particular category bar. The listing 108 includes a description column 110, a serial number column 112, a use column 114, a last known location column 116, an age column 118, a service status column 120, and a service request column 122. All of the information displayed in the listing 108 comes from data repository server 40, which forwards the data to an application server 42 that is in communication with the computer device 36 that has the user interface displaying screen shot 82c.

Description column 110 contains a brief description of each of the individual medical devices 32 within the corresponding category of category summary bar 100. In the example of FIG. 9, the description identifies the specific type of medical device (e.g. a battery of the type B-B), as well as a specific number that uniquely identifies that particular medical device (e.g. 002). The particular number, in some embodiments, is set based upon the order in which the medical facility 26 acquired those types of medical devices. For example, the first type B-B battery acquired by the medical facility is assigned number 001, the second 002, and so on. Other types of information may be displayed in description column 110 in addition to, or in lieu of, the information shown in FIG. 9.

Serial number column 112 lists the individual and unique serial numbers assigned to each medical device 32. In some embodiments, the serial numbers correspond to one of the unique identifiers discussed previously. In other embodiments, one or more of the medical devices may have both a unique serial number and another unique identifier. When another such unique identifier is used, listing 108 may be a modified to display that unique identifier as well.

Use column 114 identifies the number of times each individual one of the medical devices 32 was used in the indicated time period. As was noted previously, these criteria for determining what counts as a use of a particular medical device 32 may vary from medical device to medical device, depending upon the specific type of medical device it is. Data repository server 40 receives this use data from device data sent by each individual medical device 32 to management service 22.

The last known location column 116 indicates the last known location of each device, as well as the time at which the last known location was determined. This information is taken from the location record 80e of the digital replicas 46 of the individual medical devices 32. In the particular example shown in FIG. 9, the batteries are not equipped with sensor technology to determine their own location within a given medical facility 26. However, the batteries are configured to gather transmit their device data to a battery recharger whenever the batteries are placed in the charger. The chargers, in turn, are typically installed at fixed locations within the medical facility. The chargers receive the device data from the batteries, including the identification of the batteries, and forward this device data to local management server 34 and/or management service 22. Because the locations of the individual battery chargers are fixed, their locations can be entered into system 20, which then displays their locations as a proxy for the locations of the batteries they have charged, as shown in location column 116 of FIG. 9.

The location of the devices powered by the batteries may also be determined in a similar manner. That is, for some battery powered medical devices 32, the devices communicate their device data to their respective batteries, which then communicate the devices' data and their own data to the battery chargers, when connected. The battery chargers then communicate the devices' data, the batteries' data, and their own data to management service 22.

Age column 118 lists the age of each of the displayed medical devices 32. The age may be the age of the medical device since it was manufactured, the age of the medical device since it was refurbished, the age of the medical device 32 since it was purchased, or a combination of one or more of these.

Service status column 120 identifies whether each of the listed medical devices needs servicing soon, servicing now, replacement now, replacement soon, or no servicing or replacement in the near future (i.e. not “soon”). As with service summary field 106, service status column 120 may be configured to display those medical devices 32 needing servicing or replacement now and those needing servicing or replacement soon in different colors from those medical devices 32 that don't need servicing now or soon. In one example, service status column 120 displays those medical devices 32 needing servicing or replacement now with red type in column 120 and those medical devices 32 needing servicing or replacement soon with yellow type in column 120.

Service request column 122 provides a request service icon 124 for each of the displayed medical devices 32. The service request icon 124 provides a means by which a user can easily request servicing of any of the displayed medical devices 32. Service icon 124 also provides an indication to the user as to whether or not service has been requested. After a user presses, or otherwise selects, a service icon 124, user interface 38 changes the service request icon 124 to a different form, such as icon 124a in FIG. 9. Icon 124a indicates that service has already been requested for that particular medical device 32.

When a service request icon 124 is selected and activated by a user of system 20, computer device 36 sends a message to management service 22 indicating that service has been requested for the selected medical device 32. Management service 22 consults service status record 80j contained within the digital replica 46 for that particular medical device 32. The service status record 80j contains, among other data items, information identifying who should be notified when that particular medical device needs servicing, as well as contact information (such as an email address) for one or more individuals, groups of individuals, service centers, sales agents, technicians, or other persons or entities that should be notified when a service request is filed. Management service 22 then forwards a service message to the individuals, groups, or entities identified in the contact information. With this service message, management service 22 also forwards a set of device data about the particular device, such as the location of the device, its usage and service history, the time at which the service request was activated, manufacturing and design data about the medical device 32, and/or other information.

Batteries

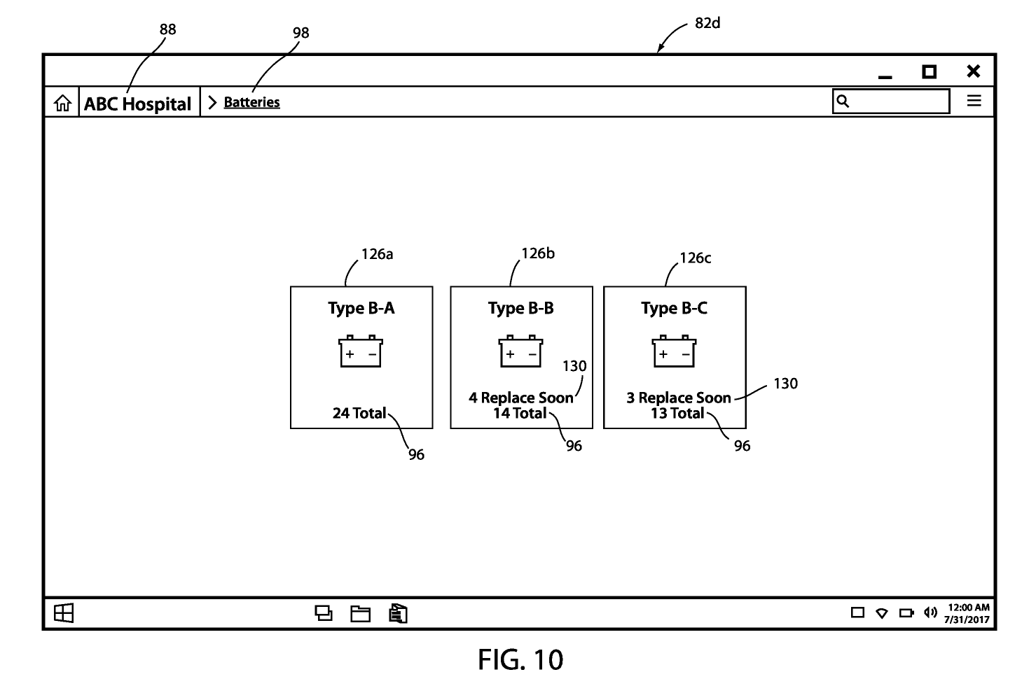

FIGS. 10-13 contain screen shots showing examples of the types of additional information that system 20 is capable of providing to a user regarding those medical devices 32 falling within category 90a, which corresponds to batteries. The screen shot 82d of FIG. 10 shows the type of information that is displayed in response to a user selecting category 90a in FIG. 8. FIG. 10 specifically identifies three subcategories 126a, 126b, and 126c within category 90a: a battery type B-A, a battery type B-B, and a battery type B-C, respectively. It will be understood that the three subcategories 126a, 126b, and 126c shown in FIG. 10 are merely representative of the number and types of subcategories 126 that may be displayed for a given category 90.

For each subcategory shown in FIG. 10, management service 22 displays a total number indicator 96 and service summary field 130. Total number indicator 96 identifies the total number of medical devices 32 in that particular subcategory that are located in the specific medical facility 26 identified by medical facility indicator 88. Thus, the three total number indicators 96 of FIG. 10 indicate that there are twenty-four type B-A batteries, fourteen type B-B batteries, and thirteen type B-C batteries.

The service summary field 130 shown in each subcategory 126 shown in FIG. 10 displays that same information shown in service summary field 106 of screen shot 82c (FIG. 9). The service summary field 106 may also use the same color scheme as that used in service summary field 106, service status column 120, and/or service request column 122 (i.e. red for those devices needing service or replacement now, yellow for those needing service or replacement soon, and black for all other medical devices). In those instances where there are no medical devices 32 within a given subcategory 126 that need servicing soon or now, user interface 38 may omit service summary field 130 or leave it blank, such as for subcategory 126a of FIG. 10.

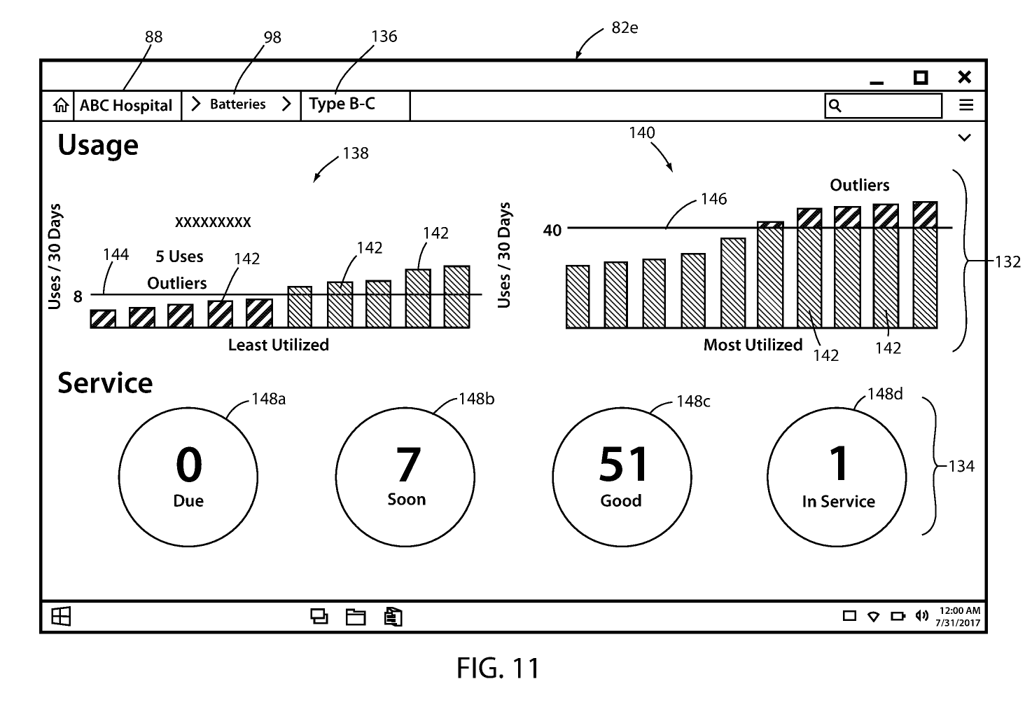

FIG. 11 illustrates a screen shot 82e that is representative of the type of data displayed on user interface 38 when a user selects one of the subcategories 126 shown in FIG. 9. Specifically, FIG. 11 illustrates a screen shot 82e representative of the type of data that may be displayed when a user selects subcategory 126b of FIG. 10. Screen shot 82e includes a usage chart area 132, a service overview area 134, a subheading identifier 136, and several components that have been previously described (e.g. facility indicator 88 and heading identifier 98).

Usage chart area 132 includes first bar chart 138 and a second bar chart 140. First bar chart 138 includes a plurality of vertical bars 142 and a horizontal threshold line 144. Each of the vertical bars 142 corresponds to a specific medical device 32 that falls within the subcategory indicated by subheading identifier 136. The height of each of the vertical bars 142 corresponds to the number of uses of that particular medical device 32 within the time period indicated along the y-axis of the 1st chart 138. First bar chart 138 illustrates vertical bars 142 for a set of the medical devices 32 within the subcategory that have had the least number of uses, or the least amount of usage (depending upon how usage is measured). In the example of FIG. 11, chart 138 illustrates the usage of the ten least utilized type B-C batteries.

The horizontal threshold line 144 is positioned along the y-axis at a value corresponding to a selected statistical cut off that provides an indication of those medical devices whose usage statistics are outliers with respect to a selected population of those medical devices within facility 26. The selected population may be all of the medical devices within facility 26, or it may be a subset of those medical devices 32 within a given facility. The selected population is at least partially selectable by one or more employees of the medical facility 26. However, an administrator of the facility 26 may limit the access of specific individuals, or classes of individuals, from seeing information regarding certain medical devices 32. Accordingly, the selected population may be all of the medical devices 32 that a particular user is allowed to see. Further, the entity that operates equipment management system 20 is also able to limit the medical devices 32 that are viewable by users of system 20, such as restricting the personnel of a first medical facility 26 from viewing status information from the medical devices 32 of a different medical facility 26 (which may be owned or operated by the same or a different entity). This may accomplished by one or more of the operator user interfaces 39.

In some situations, horizontal threshold line 144 may correspond to a value that is equal to a standard deviation below the mean usage of those medical devices. Other standard deviation multiples may be used, as well as other criteria for choosing at what value to place horizontal threshold line 144. For example, in one configuration of user interface 38, chart 138 displays horizontal threshold line 144 at a level that is equal to a fixed ranking of the medical device's usage (e.g. it is set equal to, for example, the usage of the fifth least used of the medical devices 32 within that subcategory 126). Still other criteria may be used for horizontal threshold line 144.

In some embodiments of system 20, the value at which horizontal threshold line 144 is set is user-configurable. Administrators of medical facility 26 may input whatever desired criteria or values are to be used for setting horizontal threshold line 144. Such criteria or values may be input by using any of computer devices 36 and navigating to an administrator's settings page that is displayed on user interface 38 for authorized individuals. In some configurations, the values and/or criteria used to select the values of horizontal threshold lines 144 will vary for different subcategories or categories of medical devices 32.

Regardless of the specific criteria used for choosing the location of threshold line 144, first bar chart 138 provides an easily understood graphical representation of the number of medical devices 32 within the corresponding subcategory 126 that are being underutilized. The variable heights of vertical bars 142 and their respective distances from horizontal threshold line 144 also provide a graphical indication of the amount by which those medical devices 32 are being underutilized. In some embodiments, the vertical bars 142 that are below horizontal threshold line 144 are colored differently from those that are above horizontal threshold liner 144.

Second bar chart 140 is formatted in a similar manner as first chart 138 but displays usage information at the opposite end of the usage spectrum. That is, second bar chart 140 displays information about those medical devices 32 within the particular subcategory 126 that are being overutilized. More specifically, second bar chart 140 also includes vertical bars 142 corresponding to individual medical devices 32 as well as a horizontal threshold line 146. Horizontal threshold line 146 is based on the same criteria or values used to select horizontal threshold line 144 of first chart 138, but reversed. That is, for example, if threshold line 144 represents one standard deviation below the mean usage of the medical devices 32, then threshold line 146 represents one standard deviation above the mean usage of the medical devices 32. Second chart 140 therefore provides a graphical indication of the amount by which those medical devices 32 are being overutilized. In some embodiments, the vertical bars 142 that are above horizontal threshold line 146 are colored differently from those that are below line 146.

Service overview area 134 of FIG. 11 includes four service icons 148a-d. Service icon 148a lists the number of medical devices 32 within the subcategory identified in identifier 136 (type B-C batteries in FIG. 11) that are due for service. Service icon 148b lists the number of medical devices within this subcategory that will soon need service. Service icon 148c lists the number of medical devices 32 within this subcategory that are not in need of service and are currently in an operational state. Finally, service icon 148d lists the number of medical devices 32 within this subcategory that are being serviced.

If a user wishes to view more information about which specific medical devices 32 correspond to those summarized in any of the service icons 148a, 148b, 148c, and/or 148d, he or she can click on, or otherwise select, any of the service icons 148a-d. In response, computer device 36 will display on user interface 38 a listing of the corresponding medical devices that is similar to listing 108 of FIG. 9. The displayed listing, however, unlike listing 108 of FIG. 9, will only display those medical devices 32 within the relevant subcategory that correspond to the selected service icon 148a-d (e.g. if service icon 148b is selected, the listing will only include those medical devices 32 that need service soon).

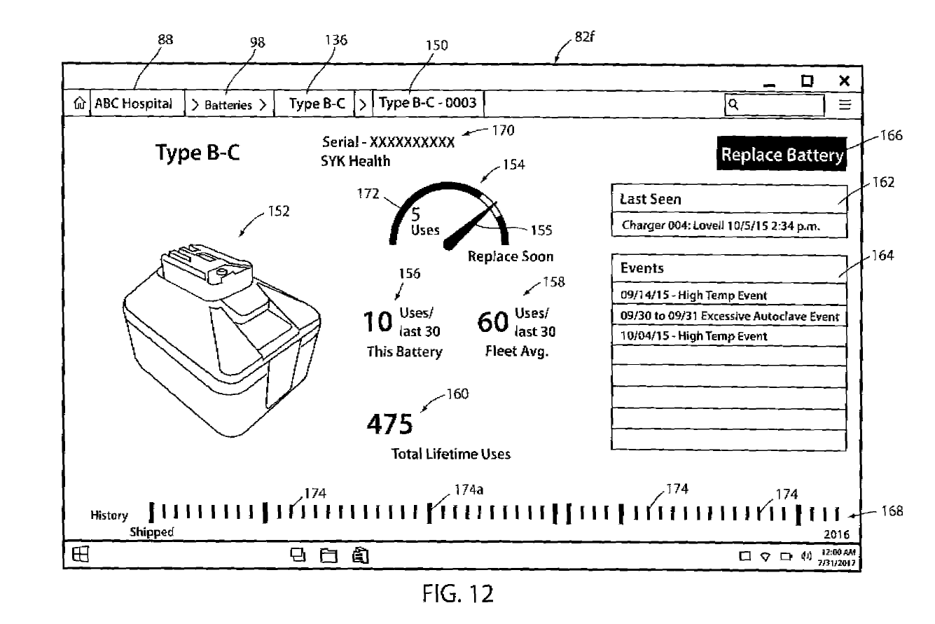

If a user wishes to see more information about any of the particular medical devices 32 represented by vertical bars 142 in charts 138 or 140, he or she can click on, or otherwise select, the vertical bar 142 and additional information will be displayed about that particular medical device, such as the information shown in FIG. 12. The type of information shown in FIG. 12 can also be accessed by other means. For example, the type of information shown in FIG. 12 can also be accessed by clicking on, or otherwise selecting, a selected row within listing 108 (FIG. 9). That is, highlighting a particular row within listing 108 and clicking on it, or otherwise selecting it, brings up a screen shot, such as screen shot 82f, that displays device data corresponding to the medical device 32 identified in the selected row.

FIG. 12 illustrates a screen shot 82f that is representative of the type of data displayed on user interface 38 when a user selects a particular medical device 32. Screen shot 82f includes a device ID heading 150, a product image 152, a health gauge 154, a use field 156, an average use field 158, a total use field 160, a last known location box 162, an event log 164, a service indicator 166, a medical device time line 168, and a serial number indicator 170.

Device ID heading 150 identifies the specific medical device 32 whose information is being displayed in screen shot 82f. Device ID heading 150 contains the same information as is found in description column 110 of listing 108. In some embodiments, device ID heading 150 contains additional information identifying the particular medical device 32 beyond what is found in column 110. In still other embodiments, device ID heading 150 contains less information than what is found in column 110. Whatever the specific content that is shown in device ID heading 150, it is based upon information stored in device ID record 80a of the device's digital replica 46.

Product image 152 is an image of the type of medical device 32 whose data is being displayed on screen shot 82f. The image displayed does not need to be an actual image of the actual medical device 32 whose specific data is displayed on screen shot 82f, but instead can be an image of that particular type of medical device 32. The image 152 is generated from the data stored in image record 80d of the digital replica 46 for that particular medical device 32. In some cases, the image is a three-dimensional image of the device that can be viewed from different angles in order to see the entire device. In other cases, the product image 152 includes one or more movies showing the image in use, or being cleaned, fixed, or maintained. In such embodiments, the user is able to select which image or movies to display in order to gain further information about the device, including how to operate, maintain, and/or service the device.

Health gauge 154 provides a graphical representation of an assessment of the health of the particular medical device whose data is shown in screen shot 82f. The graphical representation includes a pointer 155 that indicates how close the particular medical device 32 is to needing either replacement or servicing. Pointer 155 points to an arcuate scale 172 having multiple sections of different colors, wherein the different colors correspond to different qualities about the health of the medical device 32 (e.g. good, replace soon, service soon, replace now, service now, etc.). Management service 22 updates the position at which pointer 155 is displayed on arcuate scale 172 each time it receives additional device data from that particular medical device indicating it has been used further.

Use field 156 identifies the number of times the medical device 32 has been used in the time period indicated (e.g. last 30 days in FIG. 11). The information in use field 156 is the same information contained within use column 114 (see FIG. 9). It is taken from the data contained within total usage record 80i of the digital replicas 46 of the corresponding medical devices 32.

Average use field 158 identifies the average number of times the fleet of medical devices 32 have been used that are within the same subcategory as the medical device 32 whose data is shown in screen shot 82f. The time period over which this average has been taken is also displayed in average use field 158. Thus, for example, if a particular medical facility 26 has thirty five type B-C batteries, average use field 158 displays the average number of times these thirty five type B-C batteries have been used in the last thirty days. The data displayed in the average use field 158 is generated from the data contained within total usage record 80i of the corresponding digital replica 46.

Total use field 160 displays the total number of times that the particular medical device 32 has been used over its lifetime. This total use information also comes from usage record 80i of the digital replicas 46. Last known location box 162 indicates the last known location of medical device 32. The information contained within last known location box 162 is the same as that shown in last known location column 116 of listing 108.

Event log 164 lists events that have occurred with respect to the specific medical device 32 whose data is displayed on screen shot 82f. The particular events listed in event log 164 may vary for different types of medical devices 32, depending upon the design and sensing capabilities of the medical device. In the example shown in FIG. 12, event log 164 lists two high temperature events and an excessive autoclaving event. Event log 164 also lists the date for each of these events. The high temperature events are detected by one or more temperature sensors that are built into the medical device 32. The excessive autoclaving event is detected by a combination of sensors built into the medical device 32, such as one or more temperature sensors, a timer, a pressure sensor, and/or a transceiver that communicates with the autoclave.

The data displayed in event log 164 comes from event record 80k of the individual digital replicas 46. In general, the data contained in event record 80k is classified into two different categories: user-created events and device data events. The user-created events are events that are defined by a user taking one or more actions with respect to a medical device 32. For example, a user-created event includes initiating a request for maintenance or service of a device 32. A device data event refers to an event that is defined based upon the outputs of one or more sensors 58 on the medical device 32, or communications of the medical device 32 with one or more other objects or devices.

System 20 is configured such that one or more authorized users can subscribe to notifications of events when such events occur. The notifications may be via email, text, pager, phone call, or other forms of communication. The user can also specify not only what types of events he or she wishes to be notified of, but also what individual events he or she wants to be notified of. Further, different users may subscribe to notifications of different events. Thus, for example, a sales person who is not employed by medical facility 26, but who has authorized access to system 20, may use system 20 to be notified that a medical device is approaching a service date, while an administrator of the medical facility 26 may choose to use system 20 to be notified when certain medical devices 32 are finished being cleaned, or have arrived a certain location within the medical facility. A wide variety of other notification scenarios are, of course, possible.

Authorized users of system 20 can also designate events according to a priority scheme. That is, some events may be categorized as high priority while other events may be categorized as low or medium priority. Still other priority levels may be assigned, if desired. Individuals who wish to receive notifications of certain events can use the priority levels when selecting what events to be notified of. For example, a first user may only wish to receive notifications of high priority events while a second user may wish to receive notifications regarding all high and medium priority events.

Service indicator 166 provides a clear indication to the viewer of screen shot 82f of the service status of the medical device 32. The content of service indicator 166 may match the content shown in service status column 120 of FIG. 9. That is, service indicator 166 may indicate the medical device needs replacement now, will need replacement soon, needs servicing now, will need servicing soon, or will not need servicing or replacement soon. The content of service indicator 166 is generated based upon data stored in the service status record 80j of the digital replicas 46, which includes service schedules for the individual medical devices 32.

Device timeline 168 comprises a horizontal timeline having a plurality of tick marks 174. Each tick mark represents a defined time period. Some of the tick marks 174a are depicted with a larger size and/or a different color than the other tick marks 174. These tick marks 174a correspond to dates or time periods when one or more events occurred with respect to the medical device 32. By clicking on, or otherwise selecting, one of these tick marks, a user can populate event log 164 with the specific event information corresponding to that tick mark to see the details of the event(s) that occurred on that date. The data associated with time line 168 comes from the data stored in records 80e, 80f, 80g, 80j, and/or 80k of the devices' digital replicas 46.

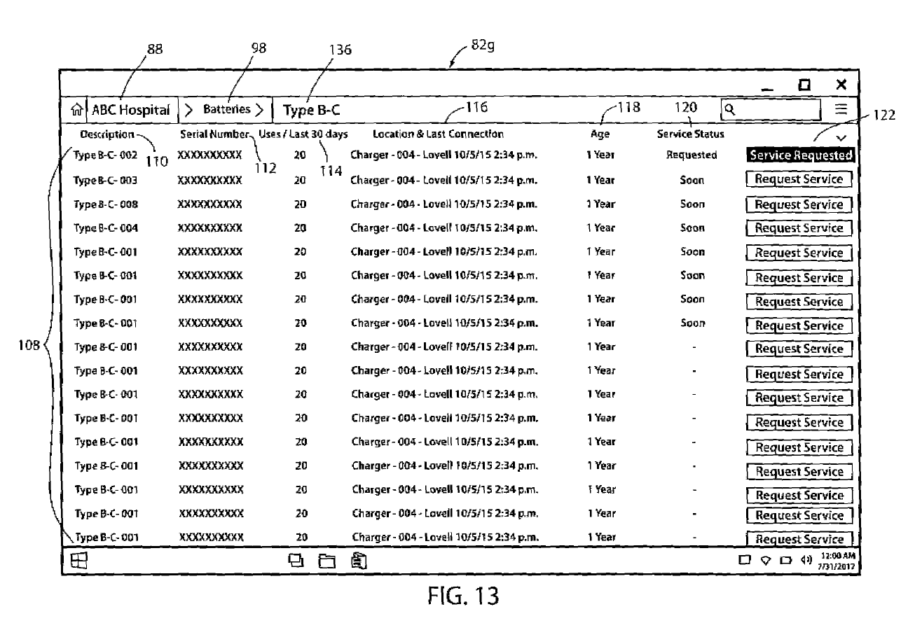

FIG. 13 illustrates a screen shot 82g that is representative of the type and layout of information that may be displayed in response to a user selecting one of the subcategories 126a, 126b, or 126c of FIG. 10. The specific example shown in FIG. 12 corresponds to a user having selected subcategory 126c, which corresponds to type B-C batteries. The type of information and overall layout of FIG. 13 is substantially the same as FIG. 9. The specific medical devices 32 identified therein, however, are different. Just as with FIG. 9, FIG. 13 includes a facility indicator 88, a heading identifier 98, a subheading identifier 136, and a listing 108. Listing 108 includes the same columns found in listing 108 of FIG. 9: a medical device description column 110, a serial number column 112, a use column 114, a last known location column 116, an age column 118, a service status column 120, and a service request column 122. These columns display the same type of data described above with respect to FIG. 9, except instead of displaying this data for all medical devices 32 as in FIG. 9, FIG. 13 displays this data for only the medical devices 32 falling within subcategory 126c. Further explanation of the layout and data displayed in FIG. 13 is therefore unnecessary.

Hand Pieces

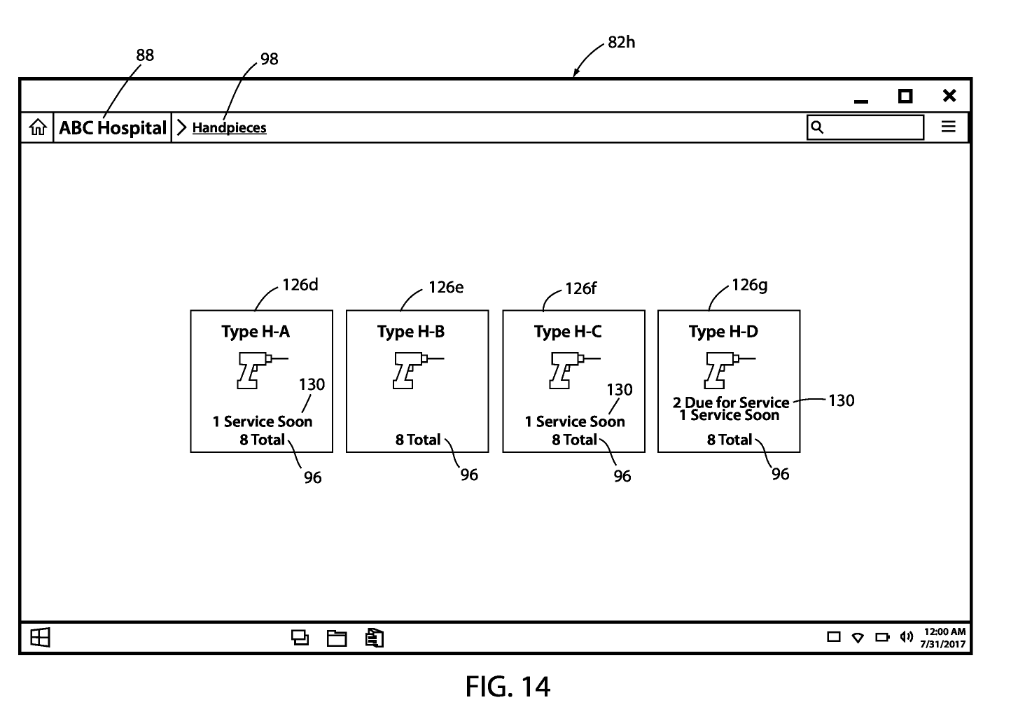

FIGS. 14-17 contain screen shots showing examples of the types of additional information that system 20 is capable of providing to a user regarding those medical devices 32 falling within category 90b, which corresponds to hand pieces. FIG. 14 shows a screen shot 82h with information of the type that is displayed in response to a user selecting category 90b in FIG. 8. That is, FIG. 14 shows all of the different subcategories 126 (if any) of the medical devices 32 within category 90b. In this particular example, FIG. 14 specifically identifies four subcategories 126d, 126e, 126f, and 126g within category 90b: a type H-A hand piece, a type H-B hand piece, a type H-C hand piece, and a type H-D hand piece, respectively. It will be understood that these four subcategories are merely representative of the number and types of subcategories 126 that may be displayed for a given category 90. Further, in some medical facilities 26, individual ones of the subcategories may be subdivided further into sub-subcategories, and so on.

As with FIG. 9, for each subcategory shown in FIG. 14, management service 22 displays a total number indicator 96 and service summary field 130. Total number indicator 96 identifies the total number of medical devices 32 in that particular subcategory that are located in the specific medical facility 26 identified by medical facility indicator 88. Thus, the total number indicators 96 of FIG. 14 indicate that there is eight of each of the four different types of identified hand pieces in Hospital ABC. The service summary field 130 shown in each subcategory 126 displays that same information shown in service summary field 106 of screen shot 82c (FIG. 9) and the service summary field 130 of FIG. 10.

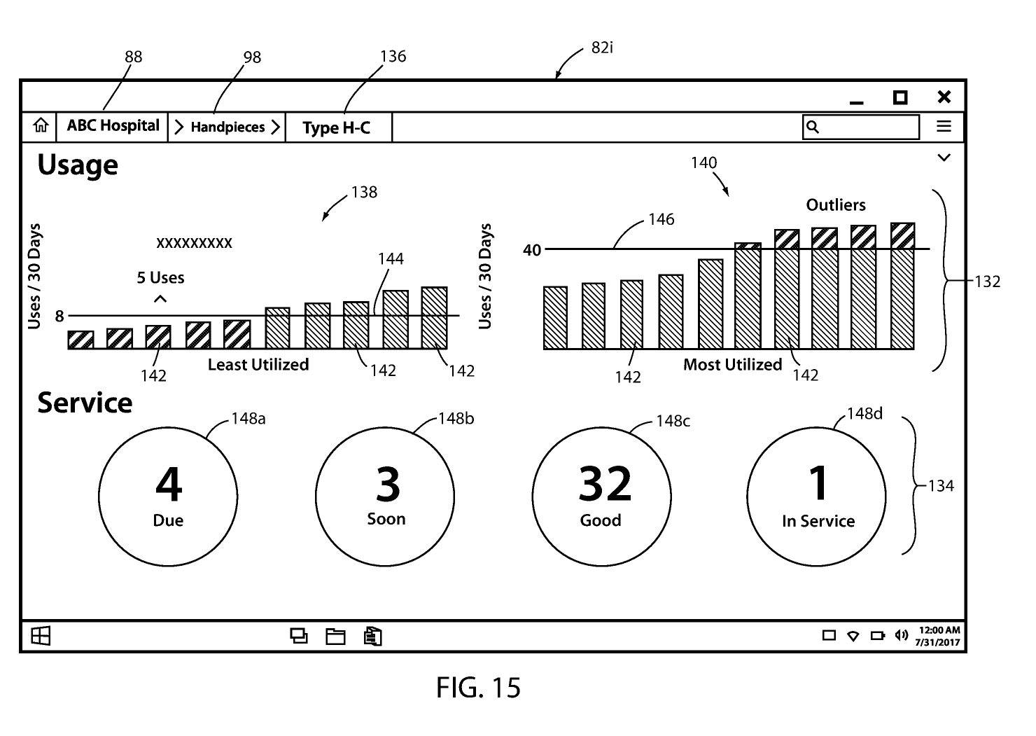

FIG. 15 illustrates a screen shot 82i that is comparable to screen shot 82e of FIG. 11, but modified to display hand piece data instead of the battery data shown in FIG. 11. Screen shot 82i of FIG. 15 is displayed in response to a user selecting subcategory 126f of FIG. 14. Like screen shot 82e of FIG. 11, FIG. 15 shows a usage chart area 132 having a first and second bar chart 138 and 140; a service overview area 134; a subheading identifier 136; a facility indicator 88; and a heading identifier 98. All of these items are the same as was previously described with respect to FIG. 11, except modified to display data corresponding to type H-C hand pieces. Accordingly, further description of FIG. 15 is not needed.

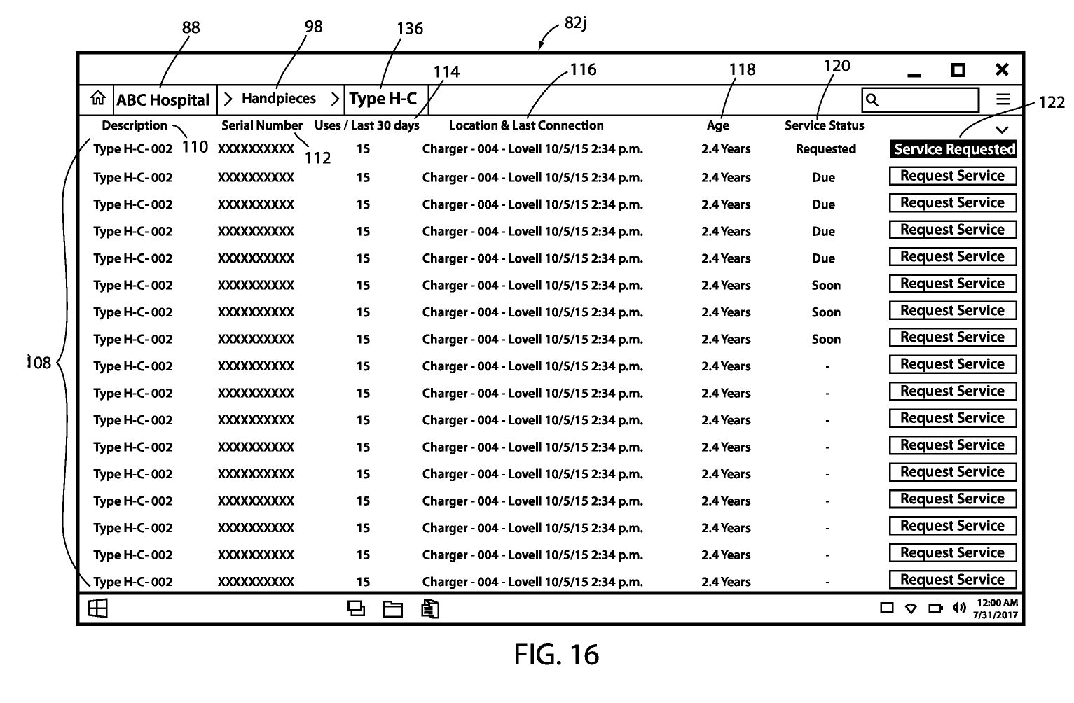

FIG. 16 illustrates a screen shot 82j that is representative of the type and layout of information that may be displayed in response to a user selecting subcategory 126f of FIG. 14, which corresponds to type H-C hand pieces. The type of information and overall layout of FIG. 16 is substantially the same as that of FIGS. 9 and 13. The specific medical devices 32 identified therein, however, are different. Just as with the screen shots of FIGS. 9 and 13, screen shot 82j of FIG. 16 includes a facility indicator 88, a heading identifier 98, a subheading identifier 136, and a listing 108. Listing 108 includes the same columns found in listings 108 of FIGS. 9 and 13: a medical device description column 110, a serial number column 112, a use column 114, a last known location column 116, an age column 118, a service status column 120, and a service request column 122. These columns display the same type of data described above with respect to FIGS. 9 and 13, except instead of displaying this data for all medical devices 32 as in FIG. 9, or data for type B-C batteries as in FIG. 13, FIG. 16 displays this data for only the medical devices 32 falling within subcategory 126f (i.e. type H-C hand pieces). Further explanation of the layout and data displayed in FIG. 16 is therefore unnecessary.

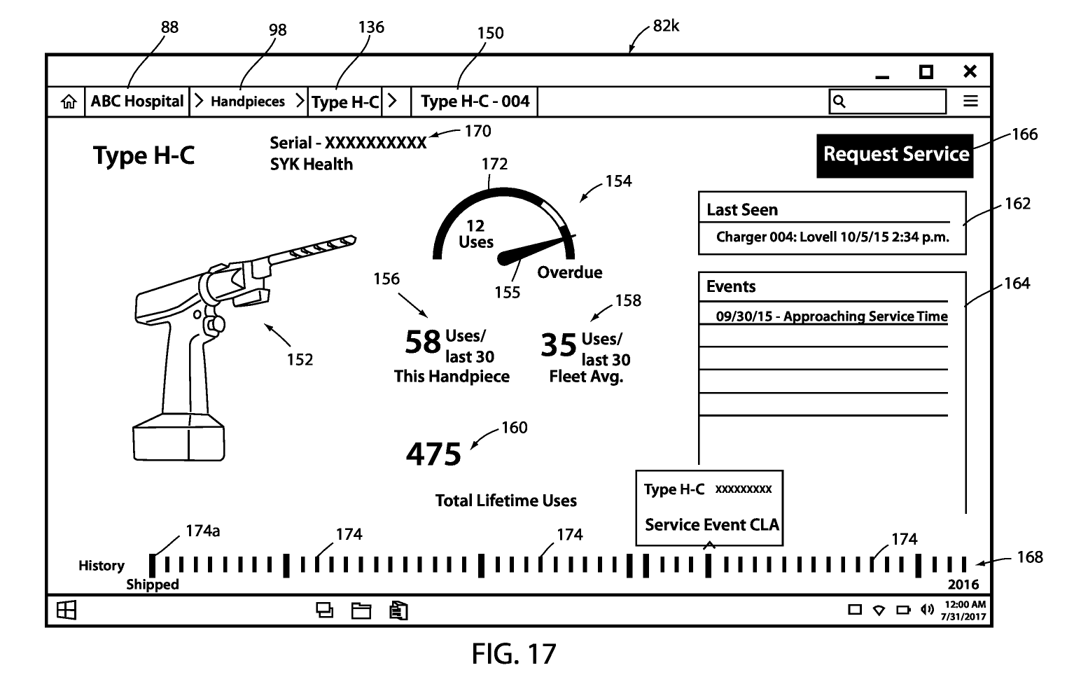

If a user wishes to see more information about any of the particular medical devices 32 represented by vertical bars 142 in charts 138 or 140 of FIG. 15, he or she can click on, or otherwise select, any of the vertical bars 142 shown therein and additional information will be displayed about the particular medical device 32 corresponding to that vertical bar 142. An example of the additional information that will be displayed is shown in FIG. 17. The information shown FIG. 17 is of the same type and layout of that shown in FIG. 12 except modified to correspond to different medical devices 32. Just as with the information of FIG. 12, the information of FIG. 17 can also be accessed by other means, such as by clicking on, or otherwise selecting, a desired row within listing 108 of FIG. 16. Further, just as with FIG. 12, FIG. 17 displays a device ID heading 150, a product image 152, a health gauge 154, a use field 156, an average use field 158, a total use field 160, a last known location box 162, an event log 164, a service indicator 166, a medical device time line 168, and a serial number indicator 170. These items provide the same functions as was previously described with respect to FIG. 12, and therefore need not be described further.

Beds

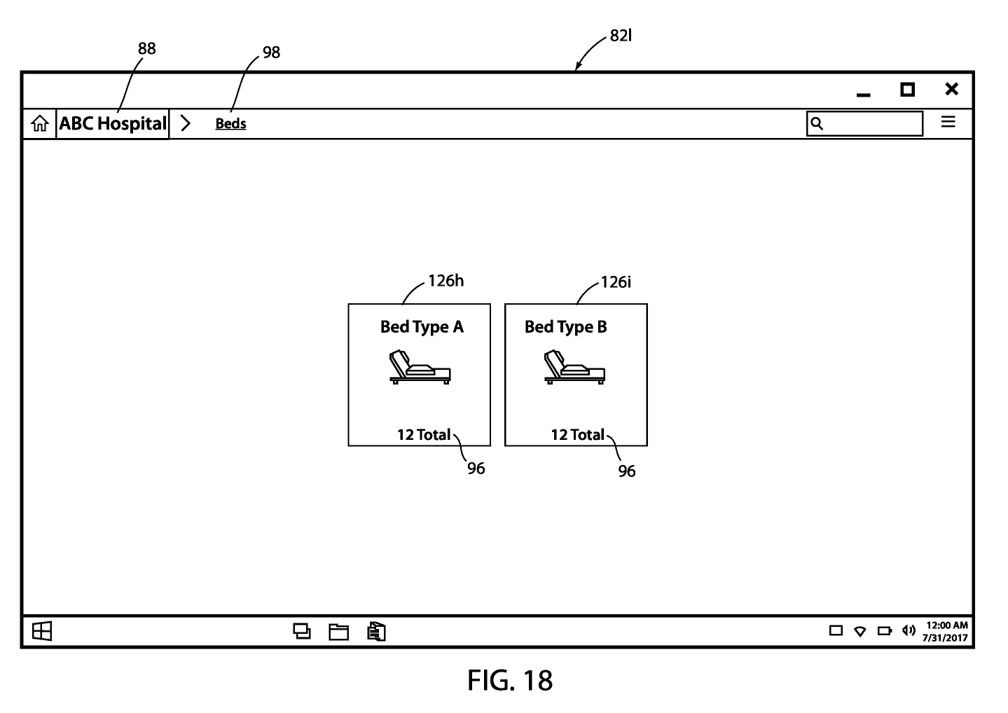

FIGS. 18-21 contain screen shots showing examples of the types of additional information that system 20 is capable of providing to a user regarding those medical devices 32 falling within category 90c, which corresponds to bed. FIG. 18 shows a screen shot 82I with information of the type that is displayed in response to a user selecting category 90c in FIG. 8. That is, FIG. 18 shows all of the different subcategories 126 (if any) of the medical devices 32 within category 90c. In this particular example, FIG. 18 specifically identifies two subcategories 126h and 126i within category 90c: a type A bed and a type B bed, respectively. It will be understood that these two subcategories are merely representative of the number and types of subcategories 126 that may be displayed for a given category 90. Further, in some medical facilities 26, individual ones of the subcategories may be subdivided further into sub-subcategories, and so on.

As with FIGS. 9 and 14, for each subcategory shown in FIG. 18, management service 22 displays a total number indicator 96 and service summary field 130. Total number indicator 96 identifies the total number of medical devices 32 in that particular subcategory that are located in the specific medical facility 26 identified by medical facility indicator 88. Thus, the total number indicators 96 of FIG. 18 indicate that there are twelve type A beds and twelve type B beds in Hospital ABC. The service summary field 130 shown in each subcategory 126 displays that same information shown in service summary field 106 of screen shot 82c (FIG. 9) and the service summary field 130 of FIG. 10. In the particular example of FIG. 18, the service summary field 130 is blank because none of the beds needs servicing now or in the near future.

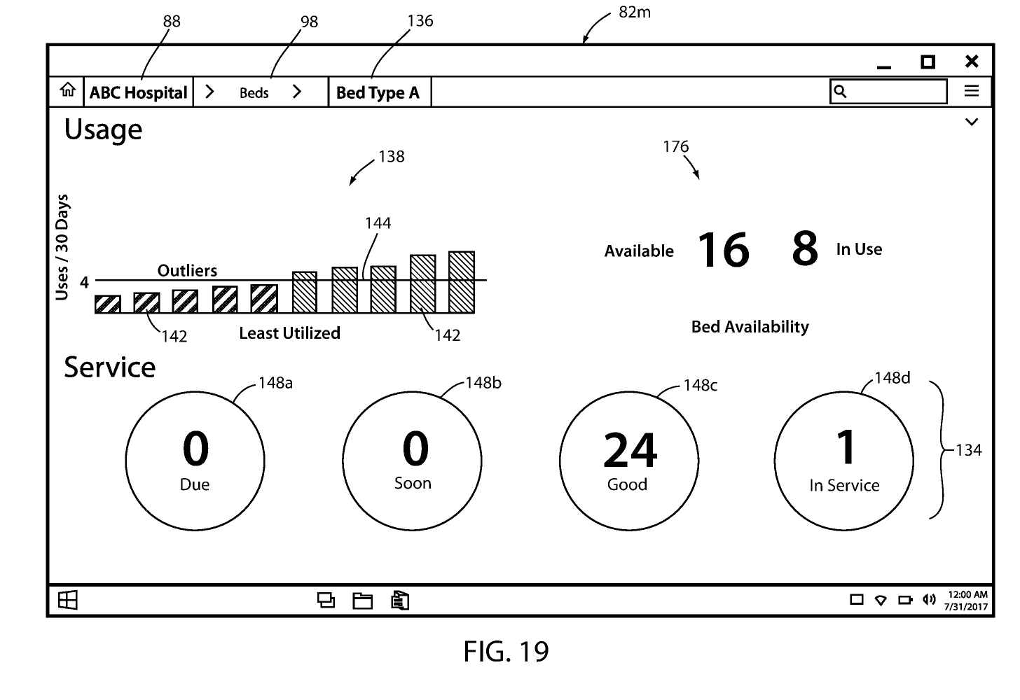

FIG. 19 illustrates a screen shot 82m that is comparable to screen shot 82e of FIGS. 11 and 82i of FIG. 15, but modified to display hand piece data instead of the battery and hand piece data shown in FIGS. 11 and 15, respectively. Screen shot 82m of FIG. 19 is displayed in response to a user selecting subcategory 126h of FIG. 18. Like screen shot 82e of FIG. 11 and screen shot 82i of FIG. 15, screen shot 82m of FIG. 19 shows a usage chart area 132 having a first bar chart 138; a service overview area 134; a subheading identifier 136; a facility indicator 88; and a heading identifier 98. All of these items are the same as was previously described with respect to FIGS. 11 and 15, except modified to display data corresponding to type A beds. Accordingly, further description of these items is not needed.

FIG. 19 differs from FIGS. 11 and 15 in that it also includes an availability indicator 176. Availability indicator 176 indicates the number of beds that are currently in use and the number of beds that are available for use. For some beds, the status of which beds are available or unavailable for use is entered into system 20 by personnel at the medical facility 26 after the bed has been cleaned and/or serviced. For other beds, the status of which beds are available or unavailable is automatically generated based on sensors incorporated into the bed. Although not shown therein, the screen shots 82e and 82i of FIGS. 11 and 15 are able to be manipulated by a user, in at least one embodiment of system 20, to also display an availability indicator 176 comparable to that shown in FIG. 19 for the medical devices 32 identified in those FIGS. Similarly, screen shot 82m of FIG. 19 can be manipulated by a user to display second bar chart 140 in place of availability indicator 176.

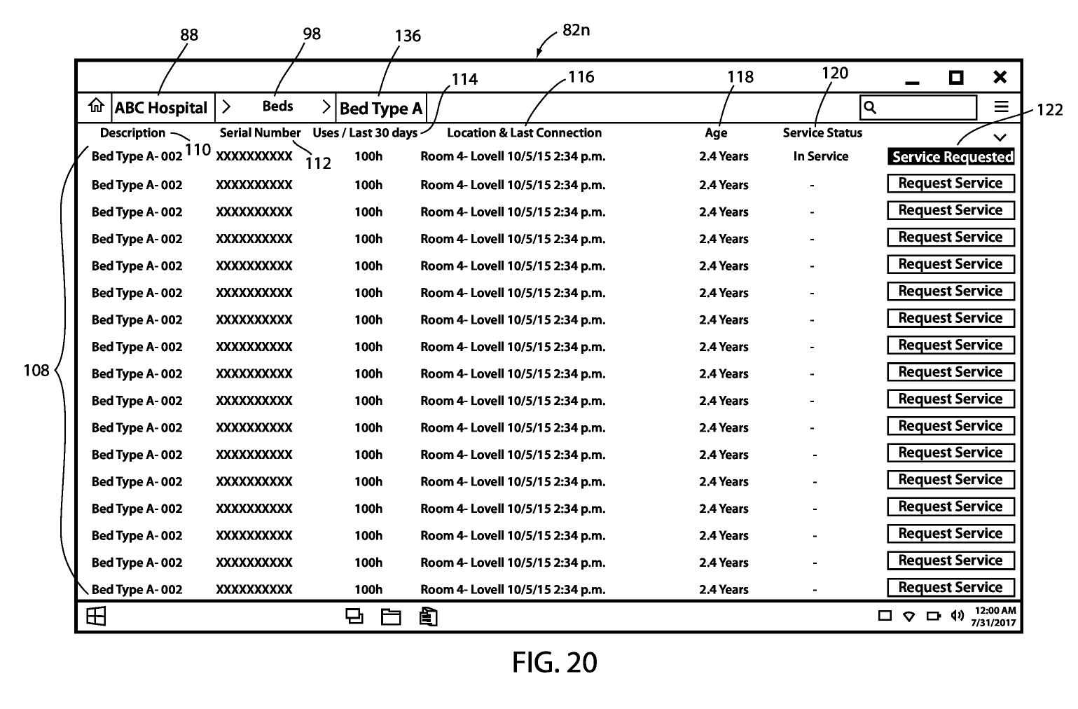

FIG. 20 illustrates a screen shot 82n that is representative of the type and layout of information that may be displayed in response to a user selecting subcategory 126h of FIG. 18, which corresponds to type A beds. The type of information and overall layout of FIG. 20 is substantially the same as that of FIGS. 9, 13, and 16. The specific medical devices 32 identified therein, however, are different. Just as with the screen shots of FIGS. 9, 13, and 16, screen shot 82n of FIG. 20 includes a facility indicator 88, a heading identifier 98, a subheading identifier 136, and a listing 108. Listing 108 includes the same columns found in listings 108 of FIGS. 9, 13, and 16: a medical device description column 110, a serial number column 112, a use column 114, a last known location column 116, an age column 118, a service status column 120, and a service request column 122. These columns display the same type of data described above with respect to FIGS. 9, 13, and 16, except that FIG. 20 displays this data for only the medical devices 32 falling within subcategory 126h (i.e. type A beds). Further explanation of the layout and data displayed in FIG. 20 is therefore unnecessary.

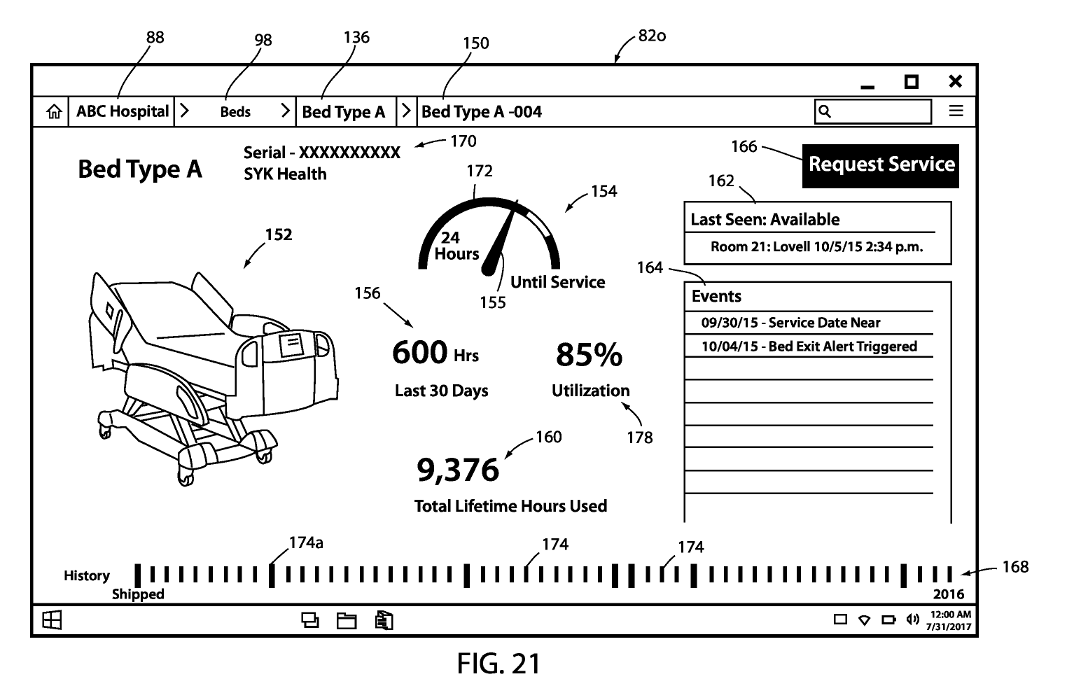

FIG. 21 illustrates a screen shot 82o that displays the same type information in the same type of layout as that of screen shots 82f and 82k of FIGS. 12 and 17, respectively, except modified to correspond to different medical devices 32. The information shown in FIG. 21 can be accessed in comparable manners to how the information of FIGS. 12 and 17 is accessed. FIG. 17 displays a device ID heading 150, a product image 152, a health gauge 154, a use field 156, a total use field 160, a last known location box 162, an event log 164, a service indicator 166, a medical device time line 168, and a serial number indicator 170. These items provide the same functions as was previously described with respect to FIGS. 12 and 17, and therefore need not be described further. FIG. 21 also includes a utilization indicator 178. Utilization indicator 178 indicates a how much time (expressed as a percentage) the bed is used compared to how much time the bed is not used.

Consoles and Other Medical Devices

FIG. 8 also includes the category 90d corresponding to consoles. When a user clicks on, or otherwise selects, category 90d of FIG. 8, user interface 38 responds by displaying screens similar to those described above for the battery, hand piece, and bed categories 90a, 90b, and 90c, respectively. It will be understood that a particular medical facility 26 may include a different set of categories than that shown in FIG. 8, including one or more categories not identified in FIG. 8. Those additional or different categories, when selected by a user, will display information comparable to what is shown in the attached drawings and described above for the battery, hand piece, and bed categories.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A medical device management system comprising:
a local network appliance at a medical facility, the local network appliance adapted to receive device data from a plurality of medical devices coupled to a local area network;
a cloud based service located remotely from the medical facility and in communication with the local network appliance, the cloud based service adapted to receive the device data from the local network appliance;
a user interface adapted to be displayed on a computer device in communication with the cloud based service, the user interface adapted to receive a data request from a user and to forward the data request to the cloud based service;
wherein the local network appliance sends location information to the cloud based service, the location information identifies the medical facility, a specific location within the medical facility, and the path by which the location information is provided between the each of the plurality of medical device and the cloud based service;
wherein each of the plurality of medical devices is configured to send an initial communication to the cloud based service which enables the cloud based service to send data back to that particular medical device; and
wherein the cloud based service is adapted to identify a set of the plurality of medical devices that are responsive to the data request and to forward to the user interface processed data regarding the set of the plurality of medical devices, the processed data being based upon the device data.

2. The medical device management system of claim 1 wherein each of the medical devices includes a transmitter and a unique identifier, each of the transmitters adapted to transmit the device data and the unique identifier to the cloud based service via the local network appliance.

3. The medical device management system of claim 1 wherein the data request requests an identification of the set of the plurality of medical devices that will need servicing within a time period.

4. (canceled)

5. The medical device management system of claim 1 wherein the user interface also forwards a user identity to the cloud based service, the cloud based service uses the user identity to filter the processed data, and the cloud based service filters the processed data by correlating the user identity to a stored profile wherein the stored profile identifies what portion of the processed data is suitable for viewing by the user.

6. The medical device management system of claim 1 where the device data includes at least one of the following: usage data indicating how long the corresponding medical device has been in use and event data indicating a time and a description of any events occurring with respect to the plurality of medical devices.

7. The medical device management system of claim 1 wherein the cloud based service updates a location record of the medical device and a last date of service record of the medical device.

8. The medical device management system of claim 7 wherein the cloud based service communicates with a sales database, the sales database including sales data identifying an entity to whom the medical devices were last sold to and a location of the entity;
the local network appliance sends location information to the cloud based service, the location information identifying the medical facility in which the medical devices are currently located; and
the cloud based service compares the entity to the identified medical facility and, if the entity does not match the identified medical facility, the cloud based service updates the location record of the medical device to correspond to the identified medical facility.

9. The medical device management system of claim 1 wherein the cloud based service includes a device management server, a data repository server, and an application server; the data repository server stores the device data and generates the processed data, the application server receives the processed data from the data repository server and forwards it to the user interface; and the local network appliance communicates with both the device management server and the data repository server, and the local network appliance forwards the device data directly to the data repository server without forwarding the device data to the device management server.

10. The medical device management system of claim 1 wherein the cloud based service communicates with a manufacturing server, the manufacturing server includes manufacturing information regarding the manufacture of the plurality of medical devices, and the processed data is based at least partially upon the manufacturing information.

11. The medical device management system of claim 1 wherein the user interface forwards the data request to the cloud based service in response to a user accessing a web page associated with the cloud based service; and wherein the user interface is adapted to allow a user to display a listing of the medical devices sorted by at least two different user-selectable criteria.

12. The medical device management system of claim 1 wherein the path by which the location information is provided comprises where a first one of medical devices is not adapted to transmit its device data directly to the local network appliance but is instead adapted to transmit its device data to a second one of the medical devices, and the second one of the medical devices is adapted to forward the device data of the first one of the medical devices to the local network appliance for forwarding to the cloud based service.

13. A medical device management system comprising:
a first local network appliance at a first medical facility and adapted to receive device data from a first set of medical devices;
a second local network appliance at a second medical facility and adapted to receive device data from a second set of medical devices;
a cloud based service located remotely from the first and second medical facilities and in communication with the first and second local network appliances, the cloud based service adapted to receive the device data from the first and second local network appliances;
a user interface adapted to be displayed on a computer device, the user interface adapted to receive a data request from a user and to forward the data request to the cloud based service;
wherein the first local network appliance and second local network appliance sends location information to the cloud based service, the location information identifies the medical facility, a specific location within the medical facility, and the path by which the location information is provided between the each medical device of the first set and the second set of medical devices and the cloud based service;
wherein each medical device of the first set and second set of medical devices is configured to send an initial communication to the cloud based service which enables the cloud based service to send data back to that particular medical device; and
wherein the cloud based service is adapted to select the first or second set of medical devices and to forward to the user interface processed data regarding the selected set of medical devices, the processed data being based upon the device data of the selected set of medical devices.

14. The medical device management system of claim 13 wherein each of the medical devices of the first and second sets of medical devices includes a unique identifier and a transmitter adapted to transmit the unique identifier and the device data and
wherein the cloud based service determines whether the computer device is associated with the first medical facility or the second medical facility, the cloud based service selects the first set of medical devices if the computer device is associated with the first medical facility and the cloud based service selects the second set of medical devices if the computer device is associated in the second medical facility.

15. (canceled)

16. The medical device management system of claim 13 wherein the user interface also forwards an access code and user identity to the cloud based service, the cloud based service uses the access code to select the first or second set of medical devices, and the cloud based service uses the user identity to select a subset of the selected set of medical devices, the cloud based service only forwarding the processed data regarding the selected subset of medical devices to the computer device.

17. The medical device management system of claim 13 wherein the device data includes usage data and repair data, the usage data indicating how long the medical devices from each of the first and second sets of medical devices have been in use and the repair data indicating when the medical devices from each of the first and second sets of medical devices were repaired; and wherein the cloud based service uses the usage data and repair data to predict when at least one of the medical devices from the first and second sets of medical devices may need repair, and the cloud based service provides notification to the user interface when the at least one of the medical devices may need repair.

18. The medical device management system of claim 13 wherein the cloud based service communicates with a sales database, the sales database including sales data identifying an entity to which the medical devices of the first and second sets of medical devices were last sold and a location of each entity;
the first and second local network appliances send location information to the cloud based service, the location information identifying the first and second medical facilities, respectively; and
the cloud based service receives device data from a first medical device from the first set of medical devices and compares the entity to whom the first medical device was sold to the first medical facility, and, if the entity to which the first medical device was sold does not match the first medical facility, the cloud based service updates a location record of the first medical device to correspond to the first medical facility.

19. A medical device management system comprising:
a local network appliance at a medical facility and adapted to receive device data from a plurality of local medical devices;
a cloud based service located remotely from the medical facility and in communication with the local network appliance, the cloud based service adapted to receive the device data from the local network appliance;
a user interface adapted to be displayed on a computer device located at the medical facility, the computer device adapted to communicate with cloud based service; and
a sales database in communication with the cloud based service, the sales database containing a first list of sales records identifying which medical devices were sold to the medical facility;
wherein each of the plurality of local medical devices is configured to send an initial communication to the cloud based service which enables the cloud based service to send data back to that particular medical device;
wherein the cloud based service is adapted to compare the first list to a second list identifying the local medical devices that are in communication with the local network appliance and to identify any differences between the first and second lists; and
wherein the computer device is adapted to display information regarding at least one of the differences.

20. (canceled)

21. The medical device management system of claim 1 wherein each of the plurality of the medical devices is configured with an initial communication parameter prior to installation at a medical facility, and wherein the cloud based service is configured to command a change in the initial communication parameter.

22. The medical device management system of claim 21 wherein the initial communication parameter is selected from among: what data is to be sent to the cloud based service, when data is to be sent to the cloud base service, a particular IP address of the cloud based service, and combinations thereof.

23. The medical device management system of claim 3, wherein the identification of the set of the plurality of medical devices that will need servicing within a time period is based upon data selected from usage data, errors, servicing requests, reported malfunctions, and combinations thereof, collected from a plurality of similar medical devices.
